(19) United States
(12) Patent Application Publication
(10) Pub. No.: US 2011/0319597 A1
(43) Pub. Date: Dec. 29, 2011

(54) VARIANT DOMAIN ANTIBODIES

(75) Inventors: Raina Jui Yu Simpson, New South Wales (AU); Zehra Elgundi, New South Wales (AU); Anthony Gerard Doyle, New South Wales (AU); Philip Anthony Jennings, New South Wales (AU); Robert Daniel Gay, New South Wales (AU); Adam William Clarke, New South Wales (AU)

(73) Assignee: Caphalon Australia Pty. Ltd., Macquarie Park (AU)

(21) Appl. No.: 13/058,422

(22) PCT Filed: Aug. 13, 2009
(86) PCT No.: PCT/AU2009/001043

§ 371 Date: May 11, 2011

Related U.S. Application Data
(60) Provisional application No. 61/089,019, filed on Aug. 14, 2008.

(30) Foreign Application Priority Data
Aug. 14, 2008 (AU) 2008904175
May 13, 2009 (AU) 2009902142

(51) Int. Cl. C07K 16/24; C12N 1/19; C12N 1/21; C12N 15/13; C12N 5/10

(52) U.S. Cl. 530/387.3; 536/23.53; 435/328; 435/252.33; 435/254.2; 435/419; 435/252.3

(57) ABSTRACT

The present invention relates to domain antibodies which have modified framework regions such that the potency or level of binding of the domain antibody is improved. In particular the present invention relates to domain antibodies which bind TNFa in which changes have been made to framework sequences and to constructs including these domain antibodies.

### FILING DATA

This application is associated with and claims priority from Australian patent application no. 2008904175 filed on 14 Aug. 2008, U.S. patent application No. 61/089,019 filed on 14 Aug. 2008 and Australian patent application no. 2009902142 filed on 13 May 2009, the entire contents of each of these applications are incorporated herein by reference.

### FIELD

The present invention relates to domain antibodies which have modified framework regions. In particular the present invention relates to domain antibodies which bind TNFα and to constructs including these domain antibodies.

### BACKGROUND

The smallest naturally-occurring functional binding component derived from an antibody is the single variable domain, also known as ‘domain antibody’ (or ‘dAb’). This may be the variable domain of the heavy or light chain of an antibody (termed ‘VH’ or ‘VL’). The functional ability of isolated variable domains was first demonstrated when a cloned repertoire of murine VH genes were expressed in bacteria and shown to interact specifically with one or more antigens (Ward et al., 1989).

Single variable-like domains occur naturally in camelids (known as VHH) and cartilaginous fish (e.g. V-NARs in sharks) mounted on Fc-equivalent constant domains (Hamers-Casterman et al., 1993, Dooley and Flajnik, 2005 and Streltsov et al., 2004) and are able to bind to cognate antigen without a complementary VL domain (Muyldermans et al., 1994). In humans, the natural occurrence of such single chain immunoglobulin molecules is linked to the unbalanced synthesis of heavy and light chains and is associated with malignant disorders, such as multiple myeloma (Jones, 1848).

Development of combinatorial libraries using variable gene chain shuffling and selection methods, such as phage display have made it possible to generate human dAb libraries from which human variable domains specific for a range of antigens have been selected. The resultant dAbs have avoided aggregation problems associated with isolating human domains that would normally be complementarily paired. Further, low immunogenicity afforded by the utilization of human variable domains has empowered the development of dAbs for therapeutic applications (Holt et al., 2003).

Domain antibodies represent good candidates for therapeutic agents as they can be engineered to specifically target molecules associated with disease. They have the additional benefits of being well expressed in bacteria, yeast and mammalian systems. Being of a small size (ranging from 11 kDa to 15 kDa), domain antibodies have potential for enhanced tissue penetration. Moreover, therapeutically important serum half lives have been engineered into domain antibody constructs (WO 04/058820A2).

Tumour necrosis factor alpha, or TNFα, is a multi-functional cytokine that plays a central role in immune regulation, infection and inflammation. While normal levels of TNFα are important to immune homeostasis, excess production of TNFα has been implicated in the pathogenesis of numerous diseases including rheumatoid arthritis, Crohn's disease and psoriasis. Blocking the activity of excess TNFα is established as an effective therapeutic strategy.

Domain antibodies targeted to human TNFα have been described. These include a human VL dAb (WO 2005/035572), VHH derived dAb (WO 2004/041862) as well as a TNFα dAb consisting of the framework of a VH domain used as a scaffold to display TNFα targeted peptides (Qin et al., 2007).

Engineering efforts aimed at improving antigen binding affinity of antibodies and dAbs are more commonly focused on hypervariable or complementarity determining regions (known as ‘CDRs’) that make specific contacts with the antigen. The CDRs form extended 0 loops that are exposed on the surface of the variable domain to provide a complementary surface for antigen binding. The three CDRs are flanked by four framework regions of conserved sequence that fold into two 13 sheets. The framework residues provide a scaffold for mounting the CDR loops and may also directly or indirectly influence antigen binding by positioning of the CDR loops (Fischmann et al., 1991 and Tulip et al., 1992). Some framework residues may directly alter the conformation of CDR loops by making atomic interactions between framework and CDR residues (Kettleborough et al., 1991, Foote and Winter, 1992 and Xiang et al., 1995).

This present invention is relevant in the field of antibody engineering. This invention particularly describes domain antibodies comprising specific framework variants that possess improved function. The invention is further relevant in the development of domain antibodies for targeting human TNFα and for the development of such domain antibodies for therapeutic applications.

Bibliographic details of the publications referred to by author in this specification are collected alphabetically at the end of the description.

Reference to any prior art in this specification is not, and should not be taken as, an acknowledgment or any form of suggestion that this prior art forms part of the common general knowledge in Australia.

### SUMMARY OF THE INVENTION

In a first aspect the present invention provides a modified dAb in which the framework sequences of the unmodified dAb are as set out in SEQ ID NO 1 and wherein the modified dAb includes at least one substitution selected from the group consisting of D1A, D1Y, D1K, I2L, I2P, Q3Y, Q3F, Q3D, Q3H, S7A, S7G, S7D, S7Q, S7N, S9A, S10A, S10L, L11Q, S12A, S12L, S12V, S12T, S12Q, S14A, D17Y, D17H, R18D, R185, T20Y, T22A, T22Y, T22Q, T22H, T22K, Q38H, P40D, K42A, K425, K42H, P44L, K45S, K45L, K45Y, K45D, K45H, 148A, Y49S, Y49D, Y49H, G57A, G57S, G57L, G57Y, G57Q, G57H, G57K, P59A, P595, P59D, P59H, S60A, S60G, S60L, S60Y, S60D, S60Q, S60H, S60P, S60K, R61H, F62Y, T69D, D70Q, L79A, L79G, L795, L79Y, L79V, L79D, L79Q, L79H, L79K, F83A, F83S, F83L, F83Y, F83D and conservative substitutions thereof and combinations thereof.

In a second aspect the present invention provides a dAb which binds TNFα, the dAb having SEQ ID NO 1 wherein at least one amino acid in the framework regions is substituted, wherein the dAb has improved TNFα neutralizing potency in comparison to the dAb of SEQ ID NO 1.

In a third aspect the present invention provides a dAb which binds TNFα, the dAb having SEQ ID NO 1 wherein at least one amino acid in the framework regions is substituted, wherein the dAb has improved TNFα binding and/or improved protein expression levels in comparison to the dAb of SEQ ID NO 1.

In a fourth aspect the present invention provides a V_{L} domain antibody comprising a V_{L} framework acceptor sequence wherein the framework sequence has been modified such that the framework comprises at least one amino acid substitution at least one position selected from the group consisting of 3, 7, 12, 60, 79, 83 and combinations thereof.

In a fifth aspect the present invention provides a nucleic acid molecule encoding the dAb of the first, second or third aspects of the present invention.

In a sixth aspect the present invention provides a transformed cell comprising the nucleic acid molecule of the fifth aspect of the present invention.

### BRIEF DESCRIPTION OF THE FIGURES

FIG. 1. TNFα binding ELISA data of Y49 variants. A representative histogram of TNFα binding ELISA showing resultant binding of variants with substitutions at position forty-nine.

FIG. 2. Representative TNFα neutralization data obtained using the high throughput 5-point format of the L929 neutralization assay.

FIG. 3. Ferritin binding ELISA data of the Ferritin dAb Fc variants. In the key, ‘Fe dAb Fc’ denotes Ferritin dAb Fc proteins.

### DETAILED DESCRIPTION

The present invention relates to dAbs in which one or more substitutions have been made in the framework region of the dAb in order to improve antigen binding efficiency, neutralizing potency or other characteristics.

In a first aspect the present invention provides a modified dAb in which the framework sequences of the unmodified dAb are as set out in SEQ ID NO 1 and wherein the modified dAb includes at least one substitution selected from the group consisting of D1A, D1Y, D1K, I2L, I2P, Q3Y, Q3F, Q3D, Q3H, S7A, S7G, S7D, S7Q, S7N, S9A, S10A, S10L, L11Q, S12A, S12L, S12V, S12T, S12Q, S14A, D17Y, D17H, R18D, R185, T20Y, T22A, T22Y, T22Q, T22H, T22K, Q38H, P40D, K42A, K42S, K42H, P44L, K45S, K45L, K45Y, K45D, K45H, I48A, Y49S, Y49D, Y49H, G57A, G57S, G57L, G57Y, G57Q, G57H, G57K, P59A, P59S, P59D, P59H, S60A, S60G, S60L, S60Y, S60D, S60Q, S60H, S60P, S60K, R61H, F62Y, T69D, D70Q, L79A, L79G, L79S, L79Y, L79V, L79D, L79Q, L79H, L79K, F83A, F83S, F83L, F83Y, F83D and conservative substitutions thereof and combinations thereof.

In the three symbol code used, the first letter stands for the amino acid in the unmodified dAb construct, the last letter stands for the substitution amino acid and the number in the middle is the residue position. For example, S60A describes a dAb variant with single substitution at position 60, where the Serine in the unmodified dAb is replaced by an Alanine. References herein to amino acid positions of unmodified dAb refer to the numbering of amino acids as defined in the Kabat database of Sequences of Proteins of Immunological Interest (“Sequences of Proteins of Immunological Interest”; US Department of Health and Human Services). The Kabat numbering is typically referred to in the art as the ‘Kabat convention’. Variants described in this invention that comprise of multiple amino acid substitutions are denoted by a series of the three symbol code separated by ‘_’. For example, S60A_F83Y is a dAb variant comprising double substitutions where the Serine and Phenylalanine that were found in positions 60 and 83 of the unmodified dAb were replaced by an Alanine and a Tyrosine, respectively.

In the sequence set out in SEQ ID NO 1 the framework regions are residues 1-23, 35-49, 57-88 and 98-108.

In various forms of the invention the modified domain antibody comprises a substitution selected from the group consisting of L79G, L79S, L79Y, L79K, L79D, L79A, L79Q, L79V and L79H; and/or a substitution selected from the group consisting of S60G, S60A, S60D, S60L, S60H, S60Q, S60Y, S60P and S60K; and/or a substitution selected from the group consisting of S12T, S12L, S12V, S12A and S12Q; and/or a substitution selected from the group consisting of F83Y, F83L, F83A, F83S and F83D; and/or a substitution selected from the group consisting of P59A, P59S, P59D and P59H; and/or a substitution selected from the group consisting of G57S, G57Q, G57H, G57A, G57L, G57Y and G57K; and/or a substitution selected from the group consisting of Q3F, Q3Y, Q3H and Q3D; and/or a substitution selected from the group consisting of S7A, S7N, S7G, S7Q and S7D.

In a second aspect the present invention provides a dAb which binds TNFα, the dAb having SEQ ID NO 1 wherein at least one amino acid in the framework regions is substituted, wherein the dAb has improved TNFα neutralizing potency in comparison to the dAb of SEQ ID NO 1.

In a preferred embodiment of the second aspect of the invention the substitution is selected from the group consisting of D1A, D1Y, D1K, I2L, I2P, Q3F, Q3Y, Q3D, Q3H, S7A, S7D, S7Q, S7G, S7N, S9A, S10A, S10L, S12A, S12L, S12Q, S12V, S12T, S14A, R18D, T22A, K42A, K45S, Y49H, G57S, G57Q, G57H, P59A, P59S, P59D, S60G, S60A, S60L, S60D, S60Q, S60H, F62Y, L79G, L79S, L79Y, L79D, L79K, F83A, F83L, F83S, F83Y and conservative substitutions thereof and combinations thereof.

In various forms of the invention the domain antibody comprises a substitution selected from the group consisting of L79G, L79S, L79Y, L79K and L79D; and/or a substitution selected from the group consisting of S60G, S60A, S60D, S60L, S60H and S60Q; and/or a substitution selected from the group consisting of S12T, S12L, S12V, S12A and S12Q; and/or a substitution selected from the group consisting of F83Y, F83L, F83A and F83S; and/or a substitution selected from the group consisting of P59A, P59S and P59D; and/or a substitution selected from the group consisting of G57S, G57Q and G57H; and/or a substitution selected from the group consisting of Q3F, Q3Y, Q3H and Q3D; and/or a substitution selected from the group consisting of S7A, S7N, S7G, S7Q and S7D.

The domain antibody may include multiple substitutions. These multiple substitutions may be selected from the group consisting of S60A_F83Y, Q3Y_S12L, S12L_S60A_L79Y_F83Y, S7Q_S60A, S7Q_S12L, Q3Y_S7Q, S12L_L79Y_F83Y, Q3Y_S60A, S12L_S60A_F83Y, S7Q_L79Y_F83Y, Q3Y_S12L_S60A_F83Y, Q3Y_S7Q_S12L_S60A, Q3Y_S60A_F83Y, Q3Y_S7Q_S12L, Q3Y_S7Q_S12L_L79Y, Q3Y_S12L_L79Y, Q3Y_L79Y, S7Q_S12L_S60A_F83Y, L79Y_F83Y, Q3Y_S12L_L79Y_F83Y, S60A_L79Y_F83Y, S7Q_S12L_L79Y_F83Y, Q3Y_S7Q_S12L_S60A_L79Y_F83Y, Q3Y_S7Q_S60A, Q3Y_S7Q_S60A_L79Y and S12L_S60A_L79Y.

In a third aspect the present invention provides a dAb which binds TNFα, the dAb having SEQ ID NO 1 wherein at least one amino acid in the framework regions is substituted, wherein the dAb has improved TNFα binding and/or improved protein expression levels in comparison to the domain antibody of SEQ ID NO 1.

In a preferred embodiment of the third aspect of the invention the substitution is selected from the group consisting of S7A, L11Q, D17Y, D17H, R185, T20Y, T22Y, T22Q, T22H, T22K, Q38H, P40D, K42S, K42H, P44L, K45L, K45Y, K45D, K45H, 148A, Y49S, Y49D, Y49H, G57A, G57S, G57L, G57Y, G57Q, G57H, G57K, P59S, P59D, P59H, S60A, S60L, S60Y, S60D, S60Q, S60H, S60P, S60K, R61H, T69D, D70Q, L79A, L79S, L79Y, L79D, L79Q, L79H, L79K, F83A, F83Y, F83D and combinations thereof.

It is preferred that the substitution is selected from the group consisting of S7A, Y49H, G57S, G57Q, G57H, P59S, P59D, S60A, S60L, S60D, S60Q, S60H, L795, L79Y, L79D, L79K, F83A, F83Y and combinations thereof.

In a fourth aspect the present invention provides a V_{L} domain antibody comprising a V_{L} framework acceptor sequence wherein the framework sequence has been modified such that the framework comprises at least one amino acid substitution at least one position selected from the group consisting of 3, 7, 12, 60, 79, 83 and combinations thereof.

In a preferred embodiment of this aspect of the present invention the domain antibody comprises at least one framework substitution selected from the group Y at position 3, Q at position 7, L at position 12, A at position 60, Y at position 79, Y at position 83 and combinations thereof.

As mentioned above the reference position number in the fourth aspect uses the Kabat numbering convention. As will be well known to persons skilled in this art a number databases providing VL and VH framework acceptor sequences are readily available. Examples of these databases include the Kabat (www.kabatdatabase.com/; Johnson and Wu, 2001), IMGT (http://imgt.cines.fr/; Lefranc, 2003) and VBase (http://vbase.mrc-cpe.cam.ac.uk/; Retter et al., 2005) databases.

In a preferred embodiment, the dAb is attached to an Fc region or a modified Fc region such as described in WO 2007/087673, (the disclosure of which is incorporated by reference) with or without the cysteine substitution. The sequence of the unmodified anti-TNFα dAb attached to the modified Fc is set out in SEQ ID NO 2. It is further preferred that the truncated CH1 and modified hinge is XEPKSZDKTHTCPPCPA (SEQ ID No: 3) wherein X is valine, leucine or isoleucine and Z is absent or an amino acid other than cysteine. In certain embodiments the modified Fc is SEQ ID NO 6 or SEQ ID NO 7.

When the dAb is attached to other domains, then the protein in its entirety is herein referred to as a ‘dAb construct’.

In a fifth aspect the present invention provides a nucleic acid molecule encoding the dAb of the first, second or third aspects of the present invention.

In a sixth aspect the present invention provides a transformed cell comprising the nucleic acid molecule of the fourth aspect of the present invention wherein the cell comprises the nucleic acid molecule.

The transformed cell may be either a prokaryotic or eukaryotic cell. Where the cell is a bacterial cell the bacteria itself may be used therapeutically as described in US 2005/0101005 and WO 2000/023471, the disclosures of which are incorporated herein by reference.

In certain embodiments the nucleic acid molecule has a sequence as set out in SEQ ID NO 8 to SEQ ID NO 708.

The present invention describes improved dAbs in which substitutions have been made in the framework regions. This highlights the importance particular framework positions play in improving potency and other characteristics. The dAbs of the present invention display improved function when substitutions at particular positions are made with amino acids of more than one amino acid class. This invention teaches the value of strong consideration of amino acid utilized at the positions of 1, 3, 7, 12, 57, 59, 60, 79 and 83 in a variable immunoglobulin domain (using the Kabat numbering convention). These positions are herein referred to as ‘robust’.

In yet another aspect, improved dAbs are variants at positions 9, 10, 12, 14, 18, 42, 79 and 83 in VL dAbs. These are dAb variants where the described framework substitutions occur at a residues whose atoms lie more than six Ångstroms (6 Å) from those CDR residues. Prior art teaches that these residues being spatially distant from CDR residues are unlikely to interact directly with the antigen, and thus unlikely to affect the interaction between the dAb and its antigen.

As described herein, a “domain antibody” or “dAb” refers to a single immunoglobulin variable domain that specifically binds a given antigen. A dAb binds antigen independently of any other domains; however, as the term is used, a dAb can be present in a homo- or heteromultimer with other domains where the other domains are not required for antigen binding by the dAb, i.e. where the dAb binds antigen independently of the additional domains. The dAb may be a heavy chain variable domain (VH) or a light chain variable domain (VL). A dAb describes the one single variable domain that is one of many domains found in an antibody molecule. The dAb of this present invention is a VL, further it is of kappa subclass (Vκ).

A domain antibody or a domain antibody construct is not an antibody. A domain antibody or dAb is one domain within an antibody. An “antibody” is a tetrameric multi-domain glycoprotein. An antibody consists of two identical pairs of immunoglobulin chains, each pair having one light and one heavy chain. The light immunoglobulin chain, which can be of either kappa or lambda subclass (Vκ or Vλ), comprises two domains, a N-terminal variable domain (VL) and one constant domain at the C-terminus (CL), while the heavy chain contains four modular domains, a N-terminal variable domain (VH) followed by three different constant domains (CH1, CH2 and CH3). The variable domains of the light and heavy chains are together responsible for binding to an antigen. The antigen binding surface of an antibody is afforded by the two variable domains, whereas that of a dAb is present within the one single domain of the dAb. It is within the scope of this present invention that the improved dAb described herein may be relevant to a subset of antibody molecules whose antigen binding surface is comprised entirely of only one of the two variable domains.

A domain antibody, whether it is in isolation, within a dAb construct or an antibody, consists of four “framework” regions separated by three hypervariable or complementarity-determining regions (or CDRs). The extents of the framework regions and CDRs in variable domains have been defined (see Kabat et al., “Sequence of Proteins of Immunological Interest”, US Department of Health and Human Services). The sequences of the framework regions of different light and heavy chains are relatively conserved within a species. Framework residues form the scaffold that positions and supports the CDRs that in turn dictate the antigen specificity and binding capacity. The CDRs are primarily responsible for antigen binding. The CDRs of each heavy and light chain are typically referred to as CDR1, CDR2 and CDR3, numbered sequentially starting from the N-terminus and are also typically identified by the chain in which the particular CDR is located. Thus, a VL CDR1 is the CDR1 from the variable domain of the light chain.

As described in this invention, “framework variants” refers herein to variable domains or dAbs which differ in amino acid sequence from an unmodified variable domain or dAb by virtue of substitution of one or more amino acid residue(s) in the unmodified sequence. The present invention describes framework variants. The variants are within the framework regions regardless of how the framework regions and CDRs are defined. For example: whether the definition utilizes the sequence-based alignment of the Kabat convention, or the structural-based definition of Chothia, or the IMGT and AHo schemes that unify the alignment of antibody immunoglobulin domains with T-cell receptor domains (Kabat et al., 1983, Chothia and Lesk, 1987, Lefranc et al., 2003 and Honegger and Pluckthun, 2001). The amino acid residue numbering used to describe the present invention follows that defined by the Kabat convention.

As used herein, a “protein” and “polypeptide” are used interchangeably and include reference to a chain of amino acid residues.

The amino acids referred herein are described: Alanine (A), Arginine (R), Asparagine (N), Aspartic Acid (D), Cysteine (C), Glutamic Acid (E), Glutamine (Q), Glycine (G), Histidine (H), Isoleucine (I), Leucine (L), Lysine (K), Methionine (M), Phenylalanine (F), Proline (P), Serine (S), Threonine (T), Tryptophan (W), Tyrosine (Y) and Valine (V).

Amino acid “substitution” refers to the replacement of the original amino acid in the unmodified protein sequence by a second amino acid at the same position in the protein sequence. The framework variants of this invention are substitution variants. That is, the specific residue or amino acid, of the unmodified variable domain or dAb is replaced by one of the other nineteen naturally occurring amino acids. This substitution does not alter the length of the polypeptide or protein sequence. The variants described in this invention are not insertion or deletion variants where the length of the polypeptide may be altered.

A substitution is deemed to be “conservative” when the original amino acid and the second or replacement amino acid have similar chemical and/or physical properties (e.g., charge, structure, polarity, hydrophobicity, hydrophilicity). As such, conservative substitutions do not substantially alter the activity of the protein. Conservative substitutions providing functionally similar amino acids are well known in the art. The following classes each contain amino acids that are conservative substitutions for one another:

(1) A, G and S

(2) N, Q, S, T, Y, K, R, H, D and E

(3) Q, N

(4) D, E

(5) K, R, H

(6) A, V, L, I, P, F, W, M, C, G

(7) Y, F, W

(8) C, S and T.

A “robust” residue or position within a protein referred herein are those residues or positions that can be substituted into amino acids of more than one amino acid class as defined above as conservative amino acids and still retain function.

The term “hotspot” means a portion of the protein sequence of a CDR or of a framework region of a variable domain which is a site of increased variation. Although CDRs are themselves considered to be regions of hypervariability, it has been learned in the present invention that particular sites, or hotspots, can be located in the framework regions which undergo certain substitution mutations.

The improved dAbs of this invention are of an immunoglobulin variable light chain domain or VL. It is of the Vκ subclass. As the invention relates to substitution of amino acids in the framework regions of the dAb and it is the CDRs, not framework residues that comprise the antigen binding surface, the dAbs described in the invention may be broadly applied to VL dAbs of all antigen specificity. The antigen may be human or non-human, protein, nucleic acid, lipid or carbohydrate.

The improved dAbs of this invention are of a VL domain of the Vκ subclass. Immunoglobulin variable light chain domains may be of either Vκ or Vλ, subclass. It is within the scope of the present invention that the improved dAbs may be also applicable in a VL dAb of lambda subclass.

The improved dAbs provided in this present invention are human dAbs. That is, the sequence of the unmodified dAb is one of a human light chain variable domain. The improved dAbs may also be applied to a humanized dAb, a CDR-grafted dAb, a synhumanised dAb, a primatized dAb, a camelid derived antibody fragment and variants thereof.

The improved dAb variants of this present invention may be utilized in isolation or be attached to other domains, e.g. comprising only a part of the protein. In a preferred embodiment of the present invention, the improved dAb is attached to a human or non-human primate heavy chain immunoglobulin constant region. The human or non-human primate heavy chain constant region may be selected from a group consisting of IgG1, IgG2, IgG3, IgG4, IgM, IgE and IgA (as well as subtypes thereof).

The improved dAb may be attached to other domains. The other attached domains may be antibody-based or antibody-like domains, such as another dAb, Fab fragments, scFv domains, VHH or V-NAR domains derived from camels, sharks and some fishes. The other domains may be non-antibody protein domains with a natural or artificially binding specificity. Naturally occurring protein domains with natural binding specificities that may be attached to the improved dAb include extracellular domains of receptors, such as those of the TNF receptor superfamily e.g. TNFRI (p55), TNFRII (p75), 95, DCR1, DCR2, DR3, DR4, DR5, DR6, EDAR, NGFR, RANK, LTβR, FN14, HVEM, CD27, CD30, CD40, 4-1BB, OX40, GITR, BCMA, TACI, BAFFR, XEDAR, TROY, RELT and soluble receptors such as osteoprotegerin and DCR3. Further others include growth hormones, glucagon-like peptide (GLP-1), interleukins (IL-2, -4, -5, -6, -7, -10, -12, -13, -14, -15, -16, -18, -21, -23, -31), FGF-basic, IGF-1, keratinocyte growth factor, insulin, LIF, GM-CSF, G-CSF, M-CSF, Epo and TPO, lymphotoxin, TRAIL, TGF-β, VEGF-2, leptin, interferons (IFN-α, -β and -γ), parathyroid hormone, Dornase alfa, TACE, thrombin, PDZ modules of signalling proteins, adhesion molecules and enzymes. Protein domains with artificially derived binding specificities include scaffolds based on transferrin (Transbody; WO 08/072,075A2), three-helix bundle from Z-domain of Protein A (Affibody®; WO 00/63243A1), human C-type lectin domain (Tetranectin; WO 98/56906A1), tenth fibronectin type III domain (AdNectin™; U.S. Pat. No. 6,818,418), Kunitz-type domain of trypsin inhibitor (WO 96/04378A2), ankyrin repeat protein, lipocalins (Anticalin®; WO 99/16873A1), 7-crystallin or ubiquitin molecule (Affilin™; WO 06/040129A2), trypsin inhibitor II (Microbody) and immunoglobulin-like domain derived from cytotoxic T lymphocyte-associated antigen 4 (CTLA-4), or modified variants thereof (Evibody™; U.S. Pat. No. 7,166,697). Further, attaching more than one domain together in a polypeptide can be achieved using linker polypeptides comprise two or more amino acid residues. Such linker polypeptides are well known in the art (see Holliger et al., 1993).

In another aspect, the improved dAbs may be conjugated to a protein such as serum albumin or a polymer (e.g. PEG). Alternatively, the dAbs may be immunoconjugated in which there is a covalent linkage of a therapeutic agent and can include cytotoxins such as native or modified Pseudomonas or Diphtheria toxin, calicheamicin and the like, encapsulating agents, (e.g. liposomes) which themselves contain pharmacological compositions, radioactive agents and other labels (e.g. enzyme reporter).

The invention relates to the specific amino acid substitution in a dAb that endows the dAb construct with improved function. The methods used to produce these mutations are wide and varied. The method utilized in the present embodiment is one of many routes that can be taken. In the case whereby the substitution(s) encompassed by the improved dAbs of this present invention are to be directly incorporated into a given human variable domain or domain antibody. The substitution may be introduced directly using standard techniques of molecular biology to prepare such DNA sequences. Substitution by site-directed mutagenesis and polymerase chain reaction (PCR) techniques is well known in the art. For example oligonucleotide directed synthesis such as that using a pre-existing variable region may be used (Jones et al., 1986, Verhoeyen et al., 1988 and Riechmann et al., 1988). Enzymatic filling of gapped nucleotide using T4 DNA polymerase may be employed (Queen et al., 1989 and WO 90/07861). This methodology will generate a small number of variants. To further increase the number of variants that can be produced at one time, one may utilize error-prone PCR to create a small library of substitution variants (Cadwell and Joyce, 1992, Hawkins et al., 1992, Fromant et al., 1995). Methods for generating high diversity libraries include DNA shuffling where recombination can be performed between variable genes or alternatively between variable gene libraries

(Stemmer et al., 1994, Harayama, 1998, Zha et al., 2003). Chain shuffling is an alternative method which involves the sequential replacement of the variable heavy and light chains (Kang et al., 1991b and Marks, 2004). In the case where large libraries of dAbs are employed for selection against a given target, natural libraries that use rearranged variable genes can be harvested from human B cells (Marks et al., 1991 and Vaughan et al., 1996) or synthetic libraries prepared from oligonucleotide cloning human immunoglobulin variable genes (Hoogenboom and Winter 1992, Barbas et al., 1992, Nissim et al., 1994, Griffiths et al., 1994 and de Kruif et al., 1995) including semi-synthetic libraries (Knappick et al., 2000).

The improved dAbs of the present invention may be identified by affinity maturation techniques. It is expected that those of skill in the art are knowledgeable in the numerous molecular engineering approaches and methods used to affinity mature antibodies and antibody-based fragments such as dAbs, Fabs, scFv and variations thereof. Typically, the CDRs are targeted in random mutagenesis (Jackson et al., 1995, van den Beucken et al., 2003, Zahnd et al., 2004, Lee et al., 2004, Yau et al., 2005) or targeted mutagenesis (Yelton et al., 1995, Schier et al., 1996, Thompson et al., 1996, Boder et al., 2005, Ho et al., 2005, Yoon et al., 2006). An example of targeted mutagenesis methodology is CDR walking involving a two-step process in which individual CDRs of the light and heavy chain are sequentially modified and the best candidates subsequently combined (Yang et al., 1995, Wu et al., 1998). Alternatively, mutations can be made across the entire sequence, for example to identify CDR and framework hotspots. This can be achieved by in vitro DNA amplification such as error-prone PCR (van den Beucken et al., 2003, Zhang et al., 2005, Thom et al., 2006, Finlay et al., 2009) or in vivo propagation of antibody genes within mutator bacterial strains (Irving et al., 1996 and Low et al., 1996, Coia et al., 2001). Targeting specific or hotspot residues for improvement can be determined experimentally using such techniques as alanine scanning (Cunnigham and Wells, 1989, Ashkenazi et al., 1990, Chatellier et al., 1995, Weiss et al., 2000, Koide et al., 2007) or homolog shotgun scanning (Vajdos et al., 2002, Murase et al., 2003, Pal et al., 2005) where the role of side chain functional groups at specific positions are identified.

When the three-dimensional structure of an antibody: antigen complex is available (e.g. obtained by x-ray crystallography), the molecular interactions between amino acids can facilitate the selection of amino acid residues for mutation. It is known that amino acid substitutions can change the three-dimensional structure of an antibody to improve function (Kast and Hilvert, 1997, Chen et al., 1999, Valjakka et al., 2002, Barderas et al., 2008). Three-dimensional models of antibodies and fragments thereof, such as domain antibodies described in this present invention, are commonly available and are familiar to those skilled in the art. Computer programs are available which illustrate and display probable three-dimensional conformational structures of selected candidate sequences. Inspection of these displays permits analysis of the likely role residues play in the recognition of the target antigen.

The improved dAbs may also be produced and selected against the target antigen using display techniques that are known to those skilled in the art. For example, bacteriophage lambda expression systems may be screened directly as bacteriophage plaques or as colonies of lysogens (Huse et al., 1989, Caton and Koprowski, 1990, Mullinax et al., 1990, Persson et al., 1991), or more commonly utilized, are selection display systems that enable a nucleic acid to be linked to the polypeptide it expresses. One of these methods is phage display techniques in which diverse protein sequences are displayed on the surface of filamentous bacteriophage, typically as fusions to bacteriophage coat proteins, or displayed externally on lambda phage capsids (phagebodies). Methods to undertake these processes are well known in the art (Scott and Smith, 1990, McCafferty et al., 1991, WO 92/01047, Kang et al., 1991a, Clackson et al., 1991, Lowman et al., 1991, Burton et al., 1991, Hoogenboom et al., 1991, Chang et al., 1991, Breitling et al., 1991, Marks et al., 1991, Barbas et al., 1992, Hawkins and Winter 1992, Marks et al., 1992, Lerner et al., 1992). Other systems for generating libraries of polypeptides involve the use of cell-free enzymatic machinery for the in vitro synthesis of the library members. For example, RNA display (Tuerk and Gold 1990, Ellington and Szostak, 1990), in vitro translation in stabilized polysome complexes (WO88/08453, WO90/05785), or in microcapsules formed by water-in-oil emulsions (WO99/02671, Tawfik and Griffiths, 1998).

The improved dAbs of this present invention may be characterized in a number of ways. One skilled in the art will appreciate the plethora of methods available. Some examples include, but are not limited to, attaching the antigen to a solid support (e.g. using an enzyme-linked immunoassay or by surface plasmon resonance analysis), in solution utilizing a labelling agent (e.g biotinylation, radiolabelling or fluorescent tagging) and employing depletion or subtraction procedures (e.g. FACS analysis; Daugherty et al., 1998, Ridgway et al., 1999, van den Beucken et al., 2003).

In another aspect, an assay for the activity or function of the target antigen may be assayed or quantified as a measurable activity or function of the target antigen. An assay for the activity or function of a target antigen includes for example, binding activity, cell activation, cell killing, cell proliferation, cell signalling, enzymatic activity, ligand-dependent internalization, promotion of cell survival and gene expression. The present invention describes framework variants that target the antigen TNFα; activity can be assayed using the TNFα-mediated cytotoxicity of L929 cells.

The term “neutralization” when used in reference to a molecule, means that the molecule interferes with a measurable activity or function of the target antigen. A molecule is neutralizing if it reduces a measurable activity or function of the target antigen. In several aspects of the present invention, where the target antigen is TNFα, neutralizing activity can be assessed using the L929 cytotoxicity assay. For example, murine L929 cells are susceptible to the cytotoxic effects of human TNFα in a dose-dependent manner. The ability to inhibit or block TNFα can be quantitated by the neutralization of the TNFα-mediated cytotoxicity of L929 cells. This ability to inhibit TNFα, or the activity as a TNFα inhibitor, is referred to as “potency”, such that the activity or potency of the improved dAbs of this invention refers to its ability to inhibit effects of TNFα in this assay.

Binding activity of the dAbs of this current invention for target antigen may be measured in a number of different assays. Such binding assays are well-known to those skilled in the art. These include enzyme-linked immunosorbent assay (ELISA) and analysis by surface plasmon resonance (SPR). Binding assays enable the measurement of the strength of the interaction between the two said molecules.

The term “dissociation constant” or “KD” refers to the strength of the interaction between two molecules, and in this invention, the affinity of dAb or dAb variant for its target antigen. The “KD” is the dissociation constant at equilibrium, and has units of Molarity. The affinity of an antibody or dAb for an antigen can be determined experimentally using any suitable method (eg. Berzofsky et al., 1984 and Kuby, 1992; and methods described herein).

As used herein “nucleic acid” or “nucleic acid molecule” includes reference to a deoxyribonucleotide or ribonucleotide polymer in either single-, double-, triple-stranded or any combination thereof. Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. Nucleic acid molecules of the present invention can include nucleic acid molecules comprising the coding sequence for a dAb, or dAb variant or specified portion; and nucleic acid molecules which comprise a nucleotide sequence different from those described above but which, due to the degeneracy of the genetic code, still encode at least one dAb or dAb variant as described herein and/or as known in the art. The genetic code is well known in the art. Thus, it would be routine for one skilled in the art to generate such degenerate nucleic acid variants that code for specific dAb or dAb variants or specified portion of the present invention.

By “transformed cell” is meant a cell that comprises the nucleic acid molecule of this present invention. Transformed cells may be prokaryotic cells such as E. coli or eukaryotic cells such as yeast or mammalian cells. The transformed cells themselves may be used therapeutically as described in US 2005/0101005 and WO 2000/023471, the disclosures of which are incorporated herein by reference.

Further, the transformed cell may be utilized in the production of the dAb or dAb variant Fc protein of the present invention. In such case, a suitable host cell system may be used for expression of the DNA sequences encoding for the improved dAbs. These may be bacteria, plant, yeast, insect and mammalian cells. It is expected that those of skill in the art are knowledgeable in the numerous expression systems for the expression of proteins including E. coli and other bacterial hosts and suitable mammalian host cells including COS-1 (e.g. ATCC CRL 1650), COS-7 (e.g. ATCC CRL-1651), HEK293, BHK21 (e.g. ATCC CRL-10), CHO (e.g. ATCC CRL 1610). BSC-1 (e.g. ATCC CRL-26) SP2/0-Ag14 (e.g. ATCC CRL 1851), HepG2 cells, YB2/0 cells, NS0, Per.C6, EBx, HeLa cells and the like, which are readily available from, for example, American Type Culture Collection (ATCC; Manassas, Va. Usa.)

Alternatively, nucleic acids of the present invention can be expressed in a host cell by turning on (by manipulation) in a host cell that contains endogenous DNA encoding an domain antibody or specified portion or variant of the present invention. Such methods are well known in the art, e.g., as described in U.S. Pat. Nos. 5,580,734, 5,641,670, 5,733,746, and 5,733,761, entirely incorporated herein by reference.

The present invention also provides a composition comprising the dAb of the present invention together with a pharmaceutically acceptable carrier.

The present invention also provides a method of treating a disorder characterized by human TNFα activity in a human subject comprising administering to the subject an effective amount of the dAb of the present invention or a composition containing such a dAb or dAb construct.

Typically the disorder characterized by human TNFα activity is selected from the group consisting of inflammation; inflammatory diseases; sepsis, including septic shock, endotoxic shock, gram negative sepsis and toxic shock syndrome; autoimmune disease, including rheumatoid arthritis, juvenile arthritis, rheumatoid spondylitis, ankylosing spondylitis, Sjögren's syndrome, osteoarthritis and gouty arthritis, allergy, multiple sclerosis, autoimmune diabetes, autoimmune uveitis, psoriasis, pemphigoid and nephrotic syndrome; inflammatory conditions of the eye, including macular degeneration, angiogenesis-related ocular disorder (in particular age-related macular degeneration), uveitis, Behçet's disease; infectious disease, including fever and myalgias due to infection and cachexia secondary to infection; graft versus host disease; tumour growth or metastasis, hematologic malignancies; pulmonary disorders including asthma, adult respiratory distress syndrome, shock lung, chronic pulmonary inflammatory disease, pulmonary sarcoidosis, pulmonary fibrosis and silicosis; inflammatory bowel disorders including Crohn's disease and ulcerative colitis; cardiac disorders, congestive heart failure; vascular disorders including Wegener's disease, giant cell arteritis; inflammatory bone disorders, central nervous system disorders such as Alzheimer's disease; peripheral nervous system disorders such as sciatica, hepatitis, coagulation disturbances, burns, reperfusion injury, endometriosis, keloid formation and scar tissue formation.

The route of administration may be intravenous, intramuscular, bolus, intraperitoneal, subcutaneous, respiratory, inhalation, topical, nasal, vaginal, rectal, buccal, sublingual, intranasal, subdermal, and transdermal.

Typically, treatment of pathologic conditions is effected by administering an effective amount or dosage of at least one dAb composition that total, on average, a range from at least about 0.01 to 500 milligrams of at least one dAb, specified portion or variant/kilogram of patient per dose, and, preferably, from at least about 0.1 to 100 milligrams of dAb, specified portion or variant/kilogram of patient per single or multiple administration, depending upon the specific activity of dAb, specified portion or variant contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved. Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually, a dosage of active ingredient can be about 0.1 to 100 mg/kg of body weight. Ordinarily, 0.1 to 50, and, preferably, 0.1 to 10 mg/kg per administration or in sustained release form is effective to obtain desired results.

As a non-limiting example, treatment of humans or animals can be provided as a one-time or periodic dosage of at least one dAb, specified portion or variant of the present invention, 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or years, or any combination thereof, using single, infusion or repeated doses.

Dosage forms (composition) suitable for internal administration generally contain from about 0.1 milligram to about 500 milligrams of active ingredient per unit or container. In these pharmaceutical compositions, the active ingredient will ordinarily be present in an amount of about 0.5-99.999% by weight based on the total weight of the composition.

For parenteral administration, the dAb, specified portion or variant can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, may also be used. The vehicle or lyophilised powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Domain antibodies of the present invention can be delivered in a carrier, as a solution, emulsion, colloid, or suspension, or as a dry powder, using any of a variety of devices and methods suitable for administration by inhalation or other modes described herein or known in the art.

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural, synthetic or semisynthetic fatty oils or fatty acids; natural, synthetic or semisynthetic mono- or di- or tri-glycerides. Parenteral administration is known in the art and includes, but is not limited to, conventional means of injections, a gas pressured needle-less injection device as described in U.S. Pat. No. 5,851,198, and a laser perforator device as described in U.S. Pat. No. 5,839,446, entirely incorporated herein by reference.

The invention further relates to the administration of at least one dAb, specified portion or variant by parenteral, topical, subcutaneous, intramuscular, intravenous, intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An dAb, specified portion or variant compositions can be prepared for use for parenteral (subcutaneous, intramuscular, intravenous, intrarticular, etc.) administration particularly in the form of liquid solutions or suspensions; for use in topical, vaginal or rectal administration particularly in semisolid forms, such as creams and suppositories; for buccal, or sublingual administration particularly in the form of tablets or capsules; or intranasally particularly in the form of powders, nasal drops or aerosols or certain agents; or transdermally particularly in the form of a gel, ointment, lotion, suspension or patch delivery system with chemical enhancers, such as dimethyl sulfoxide, to either modify the skin structure or to increase the drug concentration in the transdermal patch (Junginger et al., In “Drug Permeation Enhancement”; Hsieh, D. S., Eds., pp. 59-90 (Marcel Dekker, Inc. New York 1994, entirely incorporated herein by reference), or with oxidising agents that enable the application of formulations containing proteins and peptides onto the skin (WO 98/53847), or applications of electric fields to create transient transport pathways, such as electroporation, or to increase the mobility of charged drugs through the skin such as iontophoresis, or application of ultrasound, such as sonophoresis (U.S. Pat. Nos. 4,309,989 and 4,767,402; the above publications and patents being entirely incorporated herein by reference).

For pulmonary administration, preferably, at least one dAb, specified portion or variant composition is delivered in a particle size effective for reaching the lower airways of the lung or sinuses. According to the invention, at least one dAb, specified portion or variant can be delivered by any of a variety of inhalation or nasal devices known in the art for administration of a therapeutic agent by inhalation. These devices capable of depositing aerosolised formulations in the sinus cavity or alveoli of a patient include metered dose inhalers, nebulisers, dry powder generators, sprayers, and the like. Other devices suitable for directing the pulmonary or nasal administration of a dAb, specified portion or variants thereof are also known in the art. All such devices can use formulations suitable for the administration for the dispensing of a dAb, specified portion or variant in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulisers like AERx™ Aradigm, the Ultravent® nebuliser (Mallinckrodt), and the Acorn II® nebuliser (Marquest Medical Products; U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be representative of specific devices suitable for the practice of this invention, and are not intended as limiting the scope of the invention. Preferably, a composition comprising at least one dAb or specified portion or variant is delivered by a dry powder inhaler or a sprayer. There are several desirable features of an inhalation device for administering at least one dAb, specified portion or variant of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Formulations for oral administration of at least one dAb, specified portion or variant, rely on the co-administration of adjuvants (e.g., resorcinols and nonionic surfactants, such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant, such as magnesium stearate, paraben, preserving agent, such as sorbic acid, ascorbic acid, α-tocopherol, antioxidant, such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. Nos. 5,879,681 and 5,871,753 are used to deliver biologically active agents orally and are known in the art.

For absorption through mucosal surfaces, compositions and methods of administering at least one dAb, specified portion or variant include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. No. 5,514,670). Mucus surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration, excipients include sugars, calcium stearate, magnesium stearate, pregelinatined starch, and the like (U.S. Pat. No. 5,849,695).

For transdermal administration, the at least one dAb, specified portion or variant is encapsulated in a delivery device, such as liposomes or polymeric nanoparticles, microparticles, microcapsules, or microspheres (referred to collectively as microparticles unless otherwise stated). A number of suitable devices are known, including microparticles made of synthetic polymers, such as polyhydroxy acids, such as polylactic acid, polyglycolic acid and copolymers thereof, polyorthoesters, polyanhydrides, and polyphosphazenes, and natural polymers, such as collagen, polyamino acids, albumin and other proteins, alginate and other polysaccharides, and combinations thereof (U.S. Pat. No. 5,814,599).

It can be sometimes desirable to deliver the domain antibodies of the present invention to the subject over prolonged periods of time, for example, for periods of one week to one year from a single administration. Various slow release, depot or implant dosage forms can be utilised. For example, a dosage form can contain a pharmaceutically acceptable non-toxic salt of the compounds that has a low degree of solubility in body fluids, for example, (a) an acid addition salt with a polybasic acid, such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation, such as zinc, calcium, bismuth, barium, magnesium, aluminium, copper, cobalt, nickel, cadmium and the like, or with an organic cation formed from e.g., N,N′-dibenzyl-ethylenediamine or ethylenediamine; or (c) combinations of (a) and (b) e.g., a zinc tannate salt. Additionally, the compounds of the present invention or, preferably, a relatively insoluble salt, such as those just described, can be formulated in a gel, for example, an aluminium monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulation in a slow degrading, non-toxic, non-antigenic polymer, such as a polylactic acid/polyglycolic acid polymer, for example, as described in U.S. Pat. No. 3,773,919. The compounds or, preferably, relatively insoluble salts, such as those described above, can also be formulated in cholesterol matrix silastic pellets, particularly for use in animals. Additional slow release, depot or implant formulations, e.g., gas or liquid liposomes, are known in the literature (U.S. Pat. No. 5,770,222 and Robinson Ed., 1978).

All scientific citations, patents, patent applications and manufacturer's technical specifications referred to hereinafter are incorporated herein by reference in their entirety.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

It is to be understood that unless otherwise indicated, the subject invention is not limited to specific formulation components, manufacturing methods, dosage regimens, or the like, as such may vary. It is also to be understood that the terminology used herein is for the purpose of describing particular embodiments only and is not intended to be limiting.

It must be noted that, as used in the subject specification, the singular forms “a”, “an” and “the” include plural aspects unless the context clearly dictates otherwise. Thus, for example, reference to “a target” includes a single target, as well as two or more targets; reference to “an oligodendrocyte” includes a single oligodendrocyte, as well as two or more oligodendrocytes; reference to “the formulation” includes a single formulation, as well as two or more formulations; and so forth.

The present invention is further described by the following non-limiting Examples.

### Example 1

### Identification of Key Framework Variants

### Design of the Framework Variants

A directed mutagenesis approach was taken in order to identify key framework residues in a domain antibody construct that improved its function. Protein functions that were improved were neutralization potency, antigen binding efficiency and/or expression levels.

The unmodified domain antibody construct comprises a variable light (VL) domain antibody (dAb) targeted to human TNFα, a modified hinge region and the heavy chain constant region of human IgG1 but wherein the constant region has a truncated CH1 domain (WO 2007/087673). The unmodified dAb construct will herein be referred to as ‘TNFα dAb Fc’. TNFα dAb Fc has the sequence of SEQ. ID. NO. 2

Complementarity-determining regions (CDRs) and framework residues of the VL domain of the domain antibody Fc construct, or TNFα dAb Fc, were identified using definitions and consensus sequences of the Kabat convention (Kabat et al., “Sequence of Proteins of Immunological Interest”, US Department of Health and Human Services).

Single substitution mutations were made to the framework regions of the VL domain of TNFα dAb Fc construct. Each ‘wild-type’ residue was substituted to one of nine amino acids. The selected amino acids are representative of the major side-chain chemistries provided by the twenty amino acids. The nine amino acids and their type of side-chain functionality are:

alanine (A), small;

serine (S) and histidine (H), nucleophilic;

glutamine (Q), amide;

aspartic acid (D), acidic;

lysine (K), basic;

leucine (L) and proline (P), hydrophobic; and

tyrosine (Y), aromatic.

Residues within and some immediately adjacent to the CDRs of the VL domain were not mutated. In cases where the wild-type residue is one of the nine representative amino acids, then substitutions into the remaining eight residues were made. In total, 78 framework residues were mutated and 640 variants containing single substitution mutations were generated in VL domains of the TNFα dAb Fc construct.

The gene fragments encoding the variants were optimized for expression in mammalian cells. They were assembled from synthetic oligonucleotides and/or PCR products and cloned into a vector suitable for mammalian cell expression. The final DNA constructs were verified by sequencing.

### First Round Screen Using TNFα Binding ELISA

The framework variants containing single substitution mutations, as well as the unmodified TNFα dAb Fc construct, were assessed for their ability to bind to target antigen, human TNFα, in a TNFα binding ELISA.

The variants were transiently expressed using suspension variant of the CHOK1 cell line. In this example, small-scale transient transfections were performed using the Freestyle™ MAX CHO transfection method (Invitrogen; 2 ml transfections in 24-well plate format). Six days after transfection, conditioned medium of transfected cells was harvested by centrifugation and tested in the following ELISA.

### TNFα Binding ELISA

Ninety-six well flat-bottom plates (MaxiSorp™, Nunc™) were coated with recombinant human TNFα (CytoLab; 1 μg/ml) in carbonate coating buffer (35 mM sodium carbonate, 15 mM sodium hydrogen carbonate, pH 9.6) overnight at 4° C. The plates were washed three times in PBS containing 0.05% Tween-20 (PBS-T) then PBS. The plates were incubated in blocking solution (1% w/v BSA diluted in PBS) for 2 hours at room temperature. After washing three times in PBS-T and PBS, 50 μl of the conditioned medium and 50 μl of diluent buffer (1% w/v BSA diluted in PBS) was added to the wells and incubated for 2 hours at room temperature. The plates were washed three times in PBS-T and PBS then incubated with secondary antibody HRP goat anti-human IgG (H+L; Zymed®, diluted 1:2000 with diluent buffer). After one hour incubation at room temperature, plates were washed three times as above then 100 μl of 3,3′,5,5′tetramethylbenzidine (TMB; Sigma Aldrich®) solution added to the wells. Colorimetric development was performed at room temperature and stopped with 50 μl of 1 M hydrochloric acid and absorbance read at 450 nm.

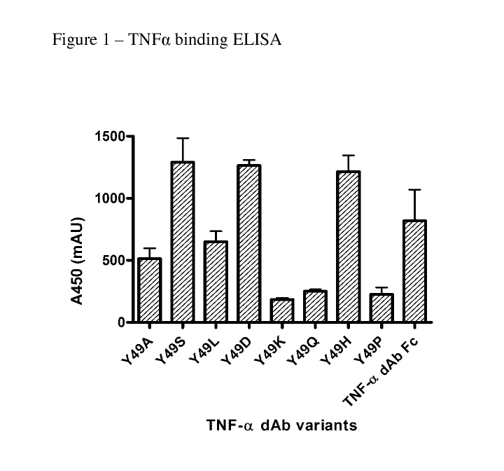

FIG. 1 is a representative graph of TNFα binding ELISA showing resultant binding of framework variants with substitutions at position forty-nine. Table 1 lists the ELISA results (A 450 nm values) for the 640 variants screened.

TABLE 1
TNFα binding ELISA data of the 640 single substitution
TNFα dAb framework variants.

| TNFα dAb | |
|---|---|
| variants | A₄₅₀ (AU) |
| D1A | 0.978 |
| D1S | 0.738 |
| D1L | 0.762 |
| D1Y | 0.971 |
| D1Q | 0.683 |
| D1H | 0.740 |
| D1P | 0.496 |
| D1K | 0.875 |
| I2A | 0.917 |
| I2S | 0.549 |
| I2L | 0.751 |
| I2Y | 0.876 |
| I2D | 0.616 |
| I2Q | 0.459 |
| I2H | 0.531 |
| I2P | 0.655 |
| I2K | 0.550 |
| Q3A | 1.057 |
| Q3S | 0.664 |
| Q3L | 0.610 |
| Q3Y | 0.963 |
| Q3D | 0.709 |
| Q3H | 0.692 |
| Q3P | 0.375 |
| Q3K | 0.589 |
| M4A | 0.289 |
| M4S | 0.230 |
| M4L | 0.731 |
| M4Y | 0.358 |
| M4D | 0.246 |
| M4Q | 0.335 |
| M4H | 0.335 |
| M4P | 0.296 |
| M4K | 0.410 |
| T5A | 0.973 |
| T5L | 0.661 |
| T5Y | 0.969 |
| T5D | 0.648 |
| T5Q | 0.685 |
| T5H | 0.703 |
| T5P | 0.333 |
| T5K | 0.643 |
| Q6A | 0.973 |
| Q6S | 0.544 |
| Q6L | 0.324 |
| Q6Y | 0.969 |
| Q6D | 0.252 |
| Q6H | 0.380 |
| Q6P | 0.262 |
| Q6K | 0.304 |
| S7A | 1.016 |
| S7L | 0.749 |
| S7Y | 0.975 |
| S7D | 0.872 |
| S7Q | 0.781 |
| S7H | 0.743 |
| S7P | 0.310 |
| S7K | 0.769 |
| P8A | 0.892 |
| P8S | 0.680 |
| P8L | 0.658 |
| P8Y | 0.694 |
| P8D | 0.564 |
| P8Q | 0.731 |
| P8H | 0.627 |
| P8K | 0.623 |
| S9A | 0.981 |
| S9L | 0.972 |
| S9Y | 0.769 |
| S9D | 0.973 |
| S9Q | 0.768 |
| S9H | 0.850 |
| S9P | 0.838 |
| S9K | 0.783 |
| S10A | 0.997 |
| S10L | 0.988 |
| S10Y | 0.795 |
| S10D | 0.813 |
| S10Q | 0.980 |
| S10H | 0.764 |
| S10P | 0.687 |
| S10K | 0.803 |
| L11A | 0.912 |
| L11S | 0.858 |
| L11Y | 0.708 |
| L11D | 0.299 |
| L11Q | 1.102 |
| L11H | 0.650 |
| L11P | 0.279 |
| L11K | 0.556 |
| S12A | 0.939 |
| S12L | 0.710 |
| S12Y | 0.704 |
| S12D | 0.636 |
| S12Q | 0.762 |
| S12H | 0.699 |
| S12P | 0.911 |
| S12K | 0.714 |
| A13S | 0.735 |
| A13L | 0.879 |
| A13Y | 0.628 |
| A13D | 0.229 |
| A13Q | 0.648 |
| A13H | 0.699 |
| A13P | 0.685 |
| A13K | 0.723 |
| S14A | 0.966 |
| S14L | 0.659 |
| S14Y | 0.937 |
| S14D | 0.787 |
| S14Q | 0.752 |
| S14H | 0.742 |
| S14P | 0.649 |
| S14K | 0.782 |
| V15A | 0.912 |
| V15S | 0.724 |
| V15Y | 0.737 |
| V15D | 0.561 |
| V15Q | 0.557 |
| V15H | 0.780 |
| V15P | 0.965 |
| V15K | 0.740 |
| G16S | 0.619 |
| G16L | 0.633 |
| G16Y | 0.942 |
| G16D | 0.542 |
| G16Q | 0.594 |
| G16H | 0.677 |
| G16P | 0.156 |
| G16K | 0.896 |
| D17A | 0.875 |
| D17S | 1.052 |
| D17L | 1.000 |
| D17Y | 1.083 |
| D17Q | 0.903 |
| D17H | 1.077 |
| D17P | 0.953 |
| D17K | 0.295 |
| R18A | 0.894 |
| R18S | 1.141 |
| R18L | 0.957 |
| R18Y | 0.900 |
| R18D | 0.984 |
| R18Q | 0.982 |
| R18H | 1.027 |
| R18P | 0.855 |
| R18K | 0.867 |
| V19A | 0.923 |
| V19S | 0.643 |
| V19Y | 0.254 |
| V19D | 0.157 |
| V19Q | 0.223 |
| V19H | 0.560 |
| V19P | 0.341 |
| V19K | 0.750 |
| T20A | 0.864 |
| T20L | 1.034 |
| T20Y | 1.119 |
| T20D | 1.043 |
| T20Q | 1.023 |
| T20H | 0.981 |
| T20P | 0.881 |
| T20K | 1.017 |
| I21A | 0.405 |
| I21S | 0.263 |
| I21L | 0.935 |
| I21Y | 0.191 |
| I21D | 0.165 |
| I21Q | 0.143 |
| I21H | 0.173 |
| I21P | 0.161 |
| I21K | 0.194 |
| T22A | 0.941 |
| T22L | 1.010 |
| T22Y | 1.036 |
| T22D | 0.985 |
| T22Q | 1.070 |
| T22H | 1.088 |
| T22P | 0.645 |
| T22K | 1.062 |
| Y36A | 0.185 |
| Y36S | 0.263 |
| Y36L | 0.237 |
| Y36D | 0.168 |
| Y36Q | 0.167 |
| Y36H | 0.149 |
| Y36P | 0.243 |
| Y36K | 0.378 |
| Q37A | 0.877 |
| Q37S | 0.962 |
| Q37L | 0.885 |
| Q37Y | 0.549 |
| Q37D | 0.251 |
| Q37H | 0.947 |
| Q37P | 0.294 |
| Q37K | 1.042 |
| Q38A | 0.783 |
| Q38S | 0.992 |
| Q38L | 0.649 |
| Q38Y | 0.793 |
| Q38D | 0.178 |
| Q38H | 1.101 |
| Q38P | 0.156 |
| Q38K | 0.909 |
| K39A | 0.802 |
| K39S | 1.004 |
| K39L | 1.056 |
| K39Y | 1.019 |
| K39D | 0.754 |
| K39Q | 0.982 |
| K39H | 1.062 |
| K39P | 0.949 |
| P40A | 0.901 |
| P40S | 1.031 |
| P40L | 1.003 |
| P40Y | 1.048 |
| P40D | 1.090 |
| P40Q | 1.053 |
| P40H | 0.990 |
| P40K | 1.041 |
| G41S | 1.062 |
| G41L | 0.906 |
| G41Y | 0.929 |
| G41D | 0.883 |
| G41Q | 1.066 |
| G41H | 0.925 |
| G41P | 0.966 |
| G41K | 0.877 |
| K42A | 0.872 |
| K42S | 1.092 |
| K42L | 0.816 |
| K42Y | 1.013 |
| K42D | 1.034 |
| K42Q | 0.903 |
| K42H | 1.074 |
| K42P | 0.246 |
| A43S | 0.831 |
| A43L | 0.718 |
| A43Y | 0.976 |
| A43D | 0.661 |
| A43Q | 1.051 |
| A43H | 1.018 |
| A43P | 0.825 |
| A43K | 1.024 |
| P44A | 0.261 |
| P44S | 0.247 |
| P44L | 1.111 |
| P44Y | 0.339 |
| P44D | 0.163 |
| P44Q | 0.182 |
| P44H | 0.309 |
| P44K | 0.187 |
| K45A | 0.865 |
| K45S | 1.038 |
| K45L | 1.112 |
| K45Y | 1.104 |
| K45D | 1.194 |
| K45Q | 1.063 |
| K45H | 1.175 |
| K45P | 0.191 |
| L46A | 0.185 |
| L46S | 0.171 |
| L46Y | 0.256 |
| L46D | 0.191 |
| L46Q | 0.234 |
| L46H | 0.166 |
| L46P | 0.179 |
| L46K | 0.243 |
| L47A | 0.257 |
| L47S | 0.222 |
| L47Y | 0.903 |
| L47D | 0.215 |
| L47Q | 0.369 |
| L47H | 0.284 |
| L47P | 0.193 |
| L47K | 0.271 |
| I48A | 1.248 |
| I48S | 0.293 |
| I48Y | 0.408 |
| I48D | 0.239 |
| I48Q | 0.265 |
| I48H | 0.234 |
| I48P | 0.193 |
| I48K | 0.253 |
| Y49A | 0.455 |
| Y49S | 1.154 |
| Y49L | 0.592 |
| Y49D | 1.233 |
| Y49Q | 0.242 |
| Y49H | 1.121 |
| Y49P | 0.188 |
| Y49K | 0.174 |
| G57A | 1.181 |
| G57S | 1.077 |
| G57L | 1.073 |
| G57Y | 1.084 |
| G57D | 0.473 |
| G57Q | 1.113 |
| G57H | 1.186 |
| G57P | 0.496 |
| G57K | 1.191 |
| V58A | 1.021 |
| V58S | 0.399 |
| V58Y | 0.247 |
| V58D | 0.185 |
| V58Q | 0.207 |
| V58H | 0.220 |
| V58P | 0.255 |
| V58K | 0.205 |
| P59A | 0.219 |
| P59S | 1.121 |
| P59L | 0.901 |
| P59Y | 0.893 |
| P59D | 1.123 |
| P59Q | 1.052 |
| P59H | 1.263 |
| P59K | 0.691 |
| S60A | 1.366 |
| S60L | 1.212 |
| S60Y | 1.352 |
| S60D | 1.208 |
| S60Q | 1.125 |
| S60H | 1.486 |
| S60P | 1.337 |
| S60K | 1.459 |
| R61A | 0.210 |
| R61S | 0.768 |
| R61L | 0.211 |
| R61Y | 0.783 |
| R61D | 0.215 |
| R61Q | 0.395 |
| R61H | 1.365 |
| R61P | 0.225 |
| F62A | 0.220 |
| F62S | 0.206 |
| F62L | 0.613 |
| F62Y | 0.960 |
| F62D | 0.208 |
| F62Q | 0.184 |
| F62H | 0.241 |
| F62P | 0.226 |
| F62K | 0.292 |
| S63A | 0.897 |
| S63L | 0.751 |
| S63Y | 0.923 |
| S63D | 0.873 |
| S63Q | 0.964 |
| S63H | 0.774 |
| S63P | 0.205 |
| S63K | 0.997 |
| G64S | 0.907 |
| G64L | 0.189 |
| G64Y | 0.193 |
| G64D | 0.171 |
| G64Q | 0.163 |
| G64H | 0.263 |
| G64P | 0.643 |
| G64K | 0.226 |
| S65A | 0.903 |
| S65L | 0.924 |
| S65Y | 1.010 |
| S65D | 0.593 |
| S65Q | 0.889 |
| S65H | 1.052 |
| S65P | 0.190 |
| S65K | 1.062 |
| G66S | 0.393 |
| G66L | 0.842 |
| G66Y | 0.909 |
| G66D | 0.808 |
| G66Q | 0.972 |
| G66H | 0.883 |
| G66P | 0.184 |
| G66K | 0.968 |
| S67A | 0.944 |
| S67L | 0.863 |
| S67Y | 0.944 |
| S67D | 1.024 |
| S67Q | 0.419 |
| S67H | 0.999 |
| S67P | 0.188 |
| S67K | 0.819 |
| G68S | 1.010 |
| G68L | 0.443 |
| G68Y | 0.305 |
| G68D | 1.002 |
| G68Q | 0.889 |
| G68H | 0.325 |
| G68P | 0.827 |
| G68K | 0.587 |
| T69A | 0.916 |
| T69L | 1.040 |
| T69Y | 1.034 |
| T69D | 1.205 |
| T69Q | 1.018 |
| T69H | 0.904 |
| T69P | 0.760 |
| T69K | 1.037 |
| D70A | 0.916 |
| D70S | 1.016 |
| D70L | 0.991 |
| D70Y | 0.938 |
| D70Q | 1.118 |
| D70H | 1.027 |
| D70P | 0.349 |
| D70K | 0.977 |
| F71A | 0.185 |
| F71S | 0.226 |
| F71L | 0.208 |
| F71Y | 0.597 |
| F71D | 0.176 |
| F71Q | 0.191 |
| F71H | 0.315 |
| F71P | 0.205 |
| F71K | 0.213 |
| T72A | 0.905 |
| T72L | 1.037 |
| T72Y | 0.193 |
| T72D | 0.916 |
| T72Q | 0.966 |
| T72H | 1.026 |
| T72P | 0.149 |
| T72K | 0.936 |
| L73A | 0.225 |
| L73S | 0.152 |
| L73Y | 0.204 |
| L73D | 0.172 |
| L73Q | 0.155 |
| L73H | 0.155 |
| L73P | 0.173 |
| L73K | 0.269 |
| T74A | 0.906 |
| T74L | 0.718 |
| T74Y | 0.640 |
| T74D | 0.962 |
| T74Q | 0.884 |
| T74H | 0.929 |
| T74P | 0.187 |
| T74K | 0.905 |
| I75A | 0.196 |
| I75S | 0.194 |
| I75L | 0.858 |
| I75Y | 0.168 |
| I75D | 0.167 |
| I75Q | 0.203 |
| I75H | 0.201 |
| I75P | 0.183 |
| I75K | 0.180 |
| S76A | 0.970 |
| S76L | 0.832 |
| S76Y | 0.797 |
| S76D | 0.758 |
| S76Q | 0.761 |
| S76H | 0.902 |
| S76P | 0.472 |
| S76K | 0.767 |
| S77A | 0.925 |
| S77L | 0.761 |
| S77Y | 0.852 |
| S77D | 0.485 |
| S77Q | 0.723 |
| S77H | 0.827 |
| S77P | 0.952 |
| S77K | 0.800 |
| L78A | 0.847 |
| L78S | 0.610 |
| L78Y | 0.379 |
| L78D | 0.176 |
| L78Q | 0.273 |
| L78H | 0.210 |
| L78P | 0.369 |
| L78K | 0.489 |
| L79V | 1.435 |
| L79P | 0.246 |
| L79A | 1.382 |
| L79G | 1.404 |
| L79S | 1.392 |
| L79Y | 1.408 |
| L79D | 1.612 |
| L79K | 1.259 |
| L79Q | 1.325 |
| L79H | 1.300 |
| P80A | 0.947 |
| P80S | 0.910 |
| P80L | 0.794 |
| P80Y | 0.766 |
| P80D | 0.741 |
| P80Q | 0.764 |
| P80H | 0.770 |
| P80K | 0.782 |
| E81A | 0.896 |
| E81S | 0.787 |
| E81L | 0.824 |
| E81Y | 0.793 |
| E81D | 0.799 |
| E81Q | 0.808 |
| E81H | 0.795 |
| E81P | 0.761 |
| E81K | 0.803 |
| D82A | 0.219 |
| D82S | 0.224 |
| D82L | 0.244 |
| D82Y | 0.265 |
| D82Q | 0.195 |
| D82H | 0.251 |
| D82P | 0.204 |
| D82K | 0.346 |
| F83A | 1.144 |
| F83G | 1.052 |
| F83S | 0.503 |
| F83L | 1.002 |
| F83Y | 1.291 |
| F83D | 1.141 |
| F83Q | 1.022 |
| F83H | 1.022 |
| F83P | 0.453 |
| A84S | 0.645 |
| A84L | 0.212 |
| A84Y | 0.179 |
| A84D | 0.177 |
| A84Q | 0.280 |
| A84H | 0.326 |
| A84P | 0.178 |
| A84K | 0.292 |
| T85A | 0.920 |
| T85L | 0.742 |
| T85Y | 0.895 |
| T85D | 0.590 |
| T85Q | 0.764 |
| T85H | 0.769 |
| T85P | 0.195 |
| T85K | 0.682 |
| Y86A | 0.153 |
| Y86S | 0.180 |
| Y86L | 0.205 |
| Y86D | 0.194 |
| Y86Q | 0.204 |
| Y86H | 0.212 |
| Y86P | 0.182 |
| Y86K | 0.185 |
| Y87A | 0.219 |
| Y87S | 0.201 |
| Y87L | 0.717 |
| Y87D | 0.208 |
| Y87Q | 0.192 |
| Y87H | 0.222 |
| Y87P | 0.176 |
| Y87K | 0.283 |
| F98A | 0.199 |
| F98S | 0.160 |
| F98L | 0.196 |
| F98Y | 0.666 |
| F98D | 0.179 |
| F98Q | 0.169 |
| F98H | 0.189 |
| F98P | 0.178 |
| F98K | 0.180 |
| G99S | 0.577 |
| G99L | 0.192 |
| G99Y | 0.213 |
| G99D | 0.177 |
| G99Q | 0.181 |
| G99H | 0.190 |
| G99P | 0.181 |
| G99K | 0.207 |
| Q100A | 0.876 |
| Q100S | 0.815 |
| Q100L | 0.628 |
| Q100Y | 0.644 |
| Q100D | 0.318 |
| Q100H | 0.729 |
| Q100P | 0.775 |
| Q100K | 0.376 |
| G101S | 0.195 |
| G101L | 0.191 |
| G101Y | 0.192 |
| G101D | 0.156 |
| G101Q | 0.199 |
| G101H | 0.214 |
| G101P | 0.961 |
| G101K | 0.295 |
| T102A | 0.383 |
| T102L | 0.210 |
| T102Y | 0.183 |
| T102D | 0.154 |
| T102Q | 0.204 |
| T102H | 0.274 |
| T102P | 0.169 |
| T102K | 0.166 |
| K103A | 0.916 |
| K103S | 1.024 |
| K103L | 0.816 |
| K103Y | 0.910 |
| K103D | 0.972 |
| K103Q | 0.258 |
| K103H | 0.975 |
| K103P | 0.319 |
| V104A | 0.667 |
| V104S | 0.208 |
| V104Y | 0.229 |
| V104D | 0.147 |
| V104Q | 0.183 |
| V104H | 0.157 |
| V104P | 0.182 |
| V104K | 0.274 |
| E105A | 0.869 |
| E105S | 0.254 |
| E105L | 0.688 |
| E105Y | 0.382 |
| E105D | 0.204 |
| E105Q | 0.412 |
| E105H | 0.506 |
| E105P | 0.237 |
| E105K | 0.612 |
| I106A | 0.867 |
| I106S | 0.172 |
| I106L | 0.165 |
| I106Y | 0.336 |
| I106D | 0.402 |
| I106Q | 0.164 |
| I106H | 0.306 |
| I106P | 0.203 |
| I106K | 0.501 |
| K106AA | 0.876 |
| K106AS | 0.294 |
| K106AL | 0.152 |
| K106AY | 0.270 |
| K106AD | 0.374 |
| K106AQ | 0.139 |
| K106AH | 0.151 |
| K106AP | 0.598 |
| R107A | 0.972 |
| R107S | 0.530 |
| R107L | 0.389 |
| R107Y | 0.395 |
| R107D | 0.161 |
| R107Q | 0.365 |
| R107H | 0.468 |
| R107P | 0.531 |
| R107K | 0.449 |
| TNFα dAb Fc | 0.819 |

This first round screen using TNFα binding ELISA identified framework variants where single substitution mutations were tolerated, i.e. variants that were expressed, secreted and retained their ability to bind to target antigen, human TNFα.

### Second Round Testing of Variants for Neutralization of TNFα Induced Cytotoxicity

A subset of TNFα dAb variants were selected and assessed for their potency in neutralizing TNFα-mediated cytotoxicity in the murine L929 cell line.

Medium-scale transfections (30 ml) were performed to obtain sufficient protein amounts for the L929 assay. The variants were transiently expressed in suspension variant of CHOK1 cell line using the Freestyle™ MAX CHO transfection method. Six days after transfection, conditioned media was harvested from the transfected cells by centrifugation and the supernatants filtered using 0.22 μm membrane filter. The TNFα dAb variants were purified by Protein A affinity chromatography. After sample loading, unbound proteins were washed off using PBS, variants were eluted using 0.1 M citric acid pH 3.5 and fractions neutralized with the addition of 1 M Tris (pH 9.0). The variants were concentrated and buffer exchanged into PBS using sample concentrators (e.g. Microcon®YM-30 units; Millipore®). Protein concentration was determined by bicinchoninic acid assay (BCA®; Pierce®).

### High Throughput 5-Point L929 Neutralization Assay

Serial half log dilutions of TNFα dAb variants in RPMI media (containing 10% foetal bovine serum and 2 mM L-glutamine) were prepared in 50 μl volume ranging from 6.25 μg/ml to 0.124 μg/ml across five wells in 96-well flat bottom plates. To each of the test wells, 25 μl of human TNFα (1.5 ng/ml), 25 μl actinomycin D (40 μg/ml) and 50 μl L929 cells (5×105 cells/ml) were added. Controls included a TNFα standard curve ranging from 3125 pg/ml to 0.172 pg/ml across eleven wells, wells containing no TNFα (100% viability) and no cells (background). Assay plates were incubated at 37° C. in a 5% CO2 humidified incubator for 20 hours then a further 2 hours after the addition of 30 μl 3-(4,5-dimethylthiazol-2-yl)-5-(3-carboxymethoxyphenyl)-2-(4-sulfophenyl)-2H-terazolium (MTS)/phenazine ethosulfate (PES). Absorbance was read at 492 nm and viability curves calculated using the average absorbance of duplicate wells. Viability curves were fitted to a sigmoidal dose model using GraphPad software (Prism®).

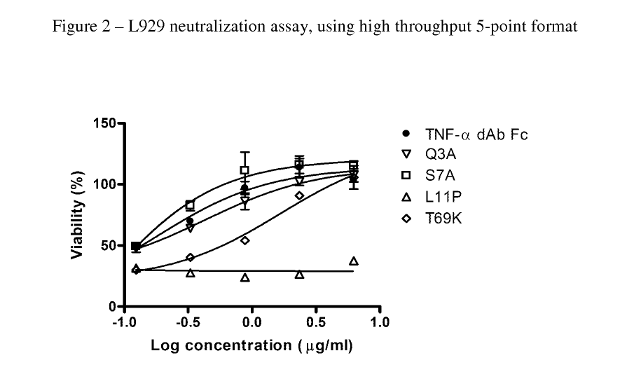

FIG. 2 is a representative result generated by the high throughput 5-point L929 assay and Table 2 presents the EC50 values of the tested TNFα dAb Fc framework variants.

TABLE 2
TNFα neutralization potency data of single substitution TNFα
dAb framework variants. Neutralization of TNFα-induced cytotoxicity
results measured as EC50 values in the L929 assay is listed. Lower
values indicate improved potency. This is an exception for those
variants where the resulting curve is a straight line as shown in
FIG. 2 for variant L11P. This data was obtained using the high throughput
5-point format of the L929 neutralization assay.

| TNFα dAb | Standardized |
|---|---|
| variants | EC50 (μg/ml) |
| D1A | 0.556 |
| D1S | 0.846 |
| D1L | 6.038 |
| D1Y | 0.676 |
| D1Q | 1.097 |
| D1H | 1.128 |
| D1K | 0.833 |
| I2A | 1.232 |
| I2S | 2.789 |
| I2L | 0.593 |
| I2Y | 3.606 |
| I2D | 3479.239 |
| I2H | 80.713 |
| I2P | 0.725 |
| I2K | 2.789 |
| Q3A | 2.323 |
| Q3S | 2.580 |
| Q3L | 6.443 |
| Q3Y | 0.450 |
| Q3D | 0.809 |
| Q3H | 0.795 |
| Q3P | 20.462 |
| Q3K | 1.819 |
| Q6A | 123.057 |
| S7A | 0.383 |
| S7L | 1.636 |
| S7Y | 0.928 |
| S7D | 0.676 |
| S7Q | 0.488 |
| S7H | 1.094 |
| S7K | 2.104 |
| S9A | 0.671 |
| S9L | 0.980 |
| S9Y | 1.996 |
| S9D | 0.880 |
| S9Q | 1.167 |
| S9H | 1.226 |
| S9P | 1.299 |
| S9K | 1.229 |
| S10A | 0.661 |
| S10L | 0.616 |
| S10Y | 1.760 |
| S10Q | 1.419 |
| S10H | 1.284 |
| S10P | 6.586 |
| S10K | 2.718 |
| L11A | 0.912 |
| L11S | 1.643 |
| L11Y | 2.774 |
| L11D | 10.150 |
| L11Q | 1.013 |
| L11P | 0.000 |
| L11K | 9.470 |
| S12A | 0.796 |
| S12L | 0.252 |
| S12Y | 1.066 |
| S12D | 1.765 |
| S12Q | 0.822 |
| S12H | 1.443 |
| S12P | 1.352 |
| S12K | 1.046 |
| S14A | 0.681 |
| S14L | 2.665 |
| S14Y | 1.924 |
| S14D | 2.262 |
| S14Q | 0.841 |
| S14H | 4134.019 |
| S14P | 9.960 |
| S14K | 1.760 |
| V15A | 2.403 |
| V15P | 1.243 |
| G16Y | 1.972 |
| D17A | 4.668 |
| D17S | 0.909 |
| D17L | 2.659 |
| D17Q | 0.863 |
| D17H | 10.542 |
| D17P | 667.543 |
| D17K | 8.844 |
| R18A | 3.208 |
| R18S | 0.851 |
| R18L | 1.000 |
| R18Y | 1.588 |
| R18D | 0.741 |
| R18Q | 2.749 |
| R18H | 2.387 |
| R18P | 1.191 |
| R18K | 0.957 |
| V19A | 1.856 |
| T20A | 1.597 |
| T20L | 1.933 |
| T20Y | 6.962 |
| T20D | 3.221 |
| T20Q | 2.020 |
| T20H | 1.686 |
| T20K | 4.381 |
| I21L | 2.395 |
| T22A | 0.725 |
| T22L | 2.509 |
| T22Y | 2.853 |
| T22D | 1.430 |
| T22Q | 2.258 |
| T22H | 11.548 |
| T22K | 2.001 |
| Q37S | 1.978 |
| Q37H | 16.398 |
| Q37K | 6.567 |
| Q38A | 9.108 |
| Q38S | 1.837 |
| Q38Y | 5045.567 |
| Q38H | 1.010 |
| Q38K | 1.890 |
| K39S | 1.601 |
| K39Q | 0.000 |
| P40A | 1.646 |
| P40S | 2.855 |
| P40L | 5.701 |
| P40Y | 26.688 |
| P40D | 1.644 |
| P40Q | 5.350 |
| P40H | 3.291 |
| P40K | 5.286 |
| G41S | 5.343 |
| G41Y | 3.569 |
| G41Q | 2.386 |
| G41H | 2.414 |
| G41P | 7.333 |
| K42A | 0.723 |
| K42S | 0.995 |
| K42L | 1.225 |
| K42Y | 3.346 |
| K42D | 1.496 |
| K42Q | 1.059 |
| K42H | 2.260 |
| A43Y | 666.360 |
| A43Q | 2.686 |
| A43H | 2.742 |
| A43K | 3.130 |
| P44A | 24.381 |
| P44S | 28.760 |
| P44L | 6.193 |
| P44Y | 27.549 |
| P44D | 802.274 |
| P44Q | 0.000 |
| P44K | 0.000 |
| K45A | 2.212 |
| K45S | 0.537 |
| K45L | 1.272 |
| K45Y | 1.377 |
| K45D | 1.404 |
| K45Q | 4.415 |
| K45H | 1.767 |
| Y49A | 6.563 |
| Y49S | 13.159 |
| Y49L | 15.836 |
| Y49D | 1.540 |
| Y49Q | 590.486 |
| Y49H | 0.731 |
| Y49P | 184.975 |
| Y49K | 1573.382 |
| G57A | 2.061 |
| G57S | 0.618 |
| G57L | 2.821 |
| G57Y | 1.005 |
| G57D | 0.948 |
| G57Q | 0.761 |
| G57H | 0.795 |
| G57P | 6.526 |
| G57K | 1.030 |
| P59A | 0.452 |
| P59S | 0.689 |
| P59L | 2.247 |
| P59Y | 1.764 |
| P59D | 0.697 |
| P59Q | 1.172 |
| P59H | 0.971 |
| P59K | 7.043 |
| S60A | 0.176 |
| S60L | 0.640 |
| S60D | 0.417 |
| S60Q | 0.746 |
| S60H | 0.735 |
| S60P | 0.895 |
| S60K | 0.867 |
| R61D | 0.000 |
| R61H | 8.868 |
| F62A | 0.000 |
| F62S | 0.000 |
| F62Y | 0.694 |
| F62D | 0.000 |
| F62H | 12.329 |
| F62P | 0.000 |
| S63Y | 2.025 |
| S63Q | 1.331 |
| S63K | 1.335 |
| G64S | 2.348 |
| S65L | 1.010 |
| S65Y | 4.725 |
| S65H | 3.713 |
| S65K | 3.493 |
| G66Y | 2.438 |
| G66Q | 2166.311 |
| G66K | 541.923 |
| S67D | 5.133 |
| G68S | 2.543 |
| G68D | 6.361 |
| T69A | 19.621 |
| T69L | 3.103 |
| T69Y | 2.490 |
| T69D | 1.345 |
| T69Q | 2.852 |
| T69K | 8.984 |
| D70A | 2.039 |
| D70S | 1.584 |
| D70Q | 3.830 |
| T72H | 5.469 |
| L79A | 1.351 |
| L79S | 0.378 |
| L79Y | 0.392 |
| L79D | 0.766 |
| L79H | 1.343 |
| L79K | 0.503 |
| F83A | 0.682 |
| F83S | 0.839 |
| F83L | 0.636 |
| F83Y | 0.493 |
| F83D | 0.947 |
| F83Q | 1.036 |
| F83H | 1.332 |
| F83P | 16.898 |
| F83G | 6.209 |
| K103S | 1.887 |
| TNFα dAb Fc | 1.200 |

The mutagenesis approach taken in the present invention identified hotspots, as well as specific substitutions, in the framework regions that resulted in increased potency of the TNFα dAb Fc variants for the TNFα antigen.

### Example 2

### TNFα dAb Variants Containing Multiple Substitutions

TNFα dAb Fc variants containing multiple substitutions in the VL domain were tested to determine whether the improved potency imparted by the single substitution variants can be combined in an additive manner. To test this, a subset of six single substitutions was selected and a panel of TNFα dAb Fc variants containing double, triple, quadruple substitutions, including a variant containing six substitution mutations were produced. The six selected substitutions are Q3Y, S7Q, S12L, S60A, L79Y and F83Y. The variants comprising multiple substitutions were produced in the same manner as the unmodified TNFα dAb Fc protein, as described in Example 1. That is, a domain antibody construct comprising a variable light (VL) domain antibody (dAb) targeted to human TNFα, a modified hinge region and the heavy chain constant region of human IgG1 but wherein the constant region has a truncated CH1 domain (WO 2007/087673). The sequence of this modified Fc is set out in SEQ ID NO. 6. The constructs described in this Example are herein collectively referred to as ‘Combination TNFα dAb framework variants’.

The panel of combination TNFα dAb framework variants were produced as described above. Briefly, gene fragments encoding the selected variants were synthesized and inserted into a vector suitable for mammalian cell expression. The variants were transiently expressed in suspension variant of the CHOK1 cell line using the Freestyle™ MAX CHO transfection method. The conditioned media was subjected to Protein A affinity chromatography, and the purified combination variants quantitated using the BCA® assay.

The combination variants were assayed for their ability to neutralize TNFα-mediated cytotoxicity using the standard 11-point format of the L929 neutralization assay.

### Standard 11-Point L929 Neutralization Assay

This assay was performed in a similar manner as described in ‘high throughput 5-point L929 neutralization assay’ (see Example 1). In this format serial half log dilutions of combination variants in RPMI media were prepared in 50 μl volume ranging from 52.5 μg/ml to 0.0005 μg/ml across eleven wells in 96-well flat bottom plates.

Table 3 presents the EC50 values of the combination TNFα dAb framework variants. Several combination variants exhibited further increases in potency, i.e. the ability to neutralize TNFα, when compared to the individual single substitution framework variants.

TABLE 3
TNFα neutralization potency data of combination TNFα
dAb framework variants, as measured by neutralization of TNFα-induced
cytotoxicity of L929 cells. Also listed are additional single substituted
TNFα dAb variants incorporating other amino acids of the same
side-chain functionalities. These are data obtained using the standard
11-point format of the L929 neutralization assay.

| | Standardized EC₅₀ (μg/ml) |
|---|---|
| Variants | Average ± S.D. |
| Q3Y | 1.198 ± 0.345 |
| Q3F | 0.910 ± 0.007 |
| S7Q | 1.496 ± 0.320 |
| S7G | 0.871 ± 0.220 |
| S7N | 0.673 ± 0.139 |
| S12L | 0.922 ± 0.269 |
| S12V | 0.979 ± 0.079 |
| S12T | 0.769 ± 0.187 |
| S60A | 0.896 ± 0.241 |
| S60E | 1.144 ± 0.183 |
| S60G | 0.644 ± 0.171 |
| L79Y | 0.950 ± 0.304 |
| L79F | 1.027 ± 0.063 |
| L79G | 0.549 ± 0.041 |
| F83Y | 0.990 ± 0.300 |
| Q3Y_S7Q | 0.922 ± 0.271 |
| Q3Y_S12L | 0.705 ± 0.011 |
| Q3Y_S60A | 0.968 ± 0.080 |
| Q3Y_L79Y | 1.205 ± 0.108 |
| Q3Y_F83Y | 2.961 ± 0.062 |
| S7Q_S12L | 0.877 ± 0.661 |
| S7Q_S60A | 0.852 ± 0.266 |
| S7Q_L79Y | 1.594 ± 0.856 |
| S7Q_F83Y | 2.240 ± 0.023 |
| S12L_S60A | 2.079 ± 0.448 |
| S12L_F83Y | 1.735 ± 0.374 |
| S60A_F83Y | 0.583 ± 0.196 |
| L79Y_F83Y | 1.292 ± 0.555 |
| Q3Y_S7Q_S12L | 1.092 ± 0.298 |
| Q3Y_S7Q_S60A | 1.431 ± 0.096 |
| Q3Y_S7Q_L79Y | 4.376 ± 3.002 |
| Q3Y_S7Q_F83Y | 2.313 ± 0.607 |
| Q3Y_S12L_S60A | 3.334 ± 1.040 |
| Q3Y_S12L_L79Y | 1.141 ± 0.125 |
| Q3Y_S12L_F83Y | 3.479 ± 0.681 |
| Q3Y_S60A_F83Y | 1.039 ± 0.441 |
| Q3Y_L79Y_F83Y | 2.408 ± 0.510 |
| S7Q_S12L_S60A | 3.539 ± 0.448 |
| S7Q_S12L_L79Y | 2.280 ± 0.078 |
| S7Q_S12L_F83Y | 2.887 ± 2.004 |
| S7Q_S60A_L79Y | 2.131 ± 0.241 |
| S7Q_S60A_F83Y | 2.243 ± 0.455 |
| S7Q_L79Y_F83Y | 1.016 ± 0.245 |
| S12L_S60A_L79Y | 1.481 ± 0.364 |
| S12L_S60A_F83Y | 0.971 ± 0.506 |
| S12L_L79Y_F83Y | 0.959 ± 0.057 |
| S60A_L79Y_F83Y | 1.367 ± 0.421 |
| Q3Y_S12L_S60A_L79Y | 1.993 ± 0.485 |
| Q3Y_S7Q_S60A_L79Y | 1.461 ± 0.475 |
| Q3Y_S7Q_S60A_F83Y | 1.982 ± 0.069 |
| Q3Y_S12L_S60A_F83Y | 1.035 ± 0.312 |
| Q3Y_S7Q_S12L_F83Y | 1.729 ± 0.186 |
| Q3Y_S60A_L79Y_F83Y | 1.966 ± 0.372 |
| Q3Y_S7Q_S12L_S60A | 1.037 ± 0.089 |
| Q3Y_S12L_L79Y_F83Y | 1.298 ± 0.513 |
| Q3Y_S7Q_L79Y_F83Y | 2.133 ± 0.038 |
| S7Q_S12L_L79Y_F83Y | 1.397 ± 0.487 |
| S7Q_S12L_S60A_F83Y | 1.281 ± 0.406 |
| S7Q_S12L_S60A_L79Y | 4.182 ± 1.036 |
| S7Q_S60A_L79Y_F83Y | 5.438 ± 2.931 |
| Q3Y_S7Q_S12L_L79Y | 1.096 ± 0.095 |
| S7Q_S12L_L79Y_F83Y | 2.795 ± 0.188 |
| S12L_S60A_L79Y_F83Y | 0.721 ± 0.235 |
| Q3Y_S7Q_S12L_S60A_L79Y_F83Y | 1.421 ± 0.245 |

### Example 3

### Testing Variants Incorporating Other Amino Acids with Same Side-Chain Functionalities

In order to ascertain whether the amino acid substitutions are representative of the other amino acids that share the same side-chain functionalities, the following additional TNFα dAb variants were tested for their TNFα neutralization potency:

(1) Q3F

(2) S7N, S7G

(3) S12V, S12T

(4) S60E, S60G

(5) L79F, L79G

These substitutions were selected as they belong to the same amino acid functional class as those identified as improved dAb variants. For example, Q3Y exhibited improved potency, therefore in this Example, Q3F was tested to determine whether the substitution of Q3 into another amino acid containing an aromatic side chain would improve potency. Variants that contain substitutions that belong to the same amino acid class as the amino acid in the original unmodified dAb construct were also tested. These are listed above as the second substitution. For example, as S7Q variant was identified as one with improved potency, S7N was tested here as Q and N are amino acids that have amide in their side-chains and further, S7G was also tested as G is an amino acid with a small side chain, like S in the unmodified construct.

These variants were produced and tested as described above. Briefly, gene fragments encoding the selected substitutions were synthesized and inserted into a vector suitable for mammalian cell expression. The variants were transiently expressed in suspension variant of the CHOK1 cell line using the Freestyle™ MAX CHO transfection method. The conditioned media was subjected to Protein A affinity chromatography and the purified protein quantitated by BCA® assay. These variants were assayed for their ability to neutralize TNFα-mediated cytotoxicity using the standard format of the L929 neutralization assay described in Example 2.

The EC50 values of variants tested in this Example are also listed in Table 3, together with data of the combination variants tested also using the standard 11-point format of the L929 assay.

This Example showed that the use of the nine amino acids as representatives of the different amino acid classes was justified (see Example 1 for the categories that the amino acids represent). All tested variants comprising substitutions into another amino acid of the same amino acid class displayed improved potency as compared to the initially identified variants, except for L79F and S60E that exhibited similar TNFα neutralization potency.

### Example 4

### Variants of Ferritin Targeted Domain Antibodies

This Example set out to test whether framework variants that resulted in improved function of the TNFα dAb can be applied to domain antibodies of different specificities.

A different domain antibody was selected—a light chain variable VL dAb targeted to human ferritin. The sequence of the anti-ferritin dAb is SEQ ID NO. 4.

In the process of designing these dAb variants, the dAb protein sequences were aligned using ClustalW (Higgins et al., 1994). Three dimensional models of VL dAbs were constructed and examined using the MODELER program (Sali and Blundell, 1993; Accelrys® Discovery Studio® v2.0.1.7347). Six framework variants that resulted in improved function of the TNFα dAb selected for this Example were position 3 to Y, position 7 to Q, 12 to L, 60 to A, 79 to Y and 83 to Y.

Ferritin VL dAb was very similar in sequence and structure, in particular in the framework region, when compared to the TNFα VL dAb. The six selected framework residues were located at similar spatial positions in both the VL dAbs, and therefore, six variants comprising of single substitution mutations at these framework regions of the ferritin dAb were made. Like the TNFα dAb variants, the ferritin VL dAb variants were also produced as Fc fusion constructs. The sequence of the unmodified ferritin VL dAb Fc construct is set out in SEQ ID NO 5. The variants produced were Q3Y, S7Q, S12L, S60A, Q79Y and F83Y and are herein collectively referred to as Territin dAb Fc variants'.

The gene fragments encoding the variants were optimized for expression in mammalian cells. They were assembled from synthetic oligonucleotides and/or PCR products and cloned into a vector suitable for mammalian cell expression. The final DNA constructs were verified by sequencing.

The Ferritin dAb Fc framework variants were transiently expressed in suspension variant of the CHOK1 cell line. Transfections were performed using Freestyle™ MAX CHO transfection method. Six days after transfection conditioned media was harvested from by centrifugation and the supernatants filtered using 0.22 μm membrane filter. The variants were purified by Protein A affinity chromatography. After sample loading, unbound proteins were washed off using PBS, variants were eluted using 0.1 M citric acid pH 3.5 and fractions neutralized with the addition of 1 M Tris pH 9.0 then concentrated and buffer exchanged into PBS using sample concentrators (namely Micron YM-30 units or Amicon® Ultra-4 devices; Millipore®). Protein concentration was determined by BCA® assay.

### Testing of Ferritin VL dAb Variants

The six Ferritin dAb Fc framework variants (Q3Y, S7Q, S12L, S60A, Q79Y and F83Y) as well as unmodified Ferritin dAb Fc construct were assessed for their ability to bind target antigen, human spleen ferritin, in a ferritin binding ELISA.

### Ferritin Binding ELISA

Ninety-six well flat-bottom plates (MaxiSorp™, Nunc™) were coated with Ferritin dAb Fc proteins (10 μg/ml) in carbonate coating buffer overnight at 4° C. After the overnight incubation, the plates were washed three times in PBS-T then PBS followed by incubation in blocking solution (1% w/v BSA diluted in PBS) for 1 hour at room temperature. After washing, human spleen ferritin (AppliChem) serially diluted 1 in 2 from 126 μg/ml to 0.246 μg/ml in diluent buffer was added to the wells and incubated for 2 hours at room temperature. The plates were washed in PBS-T and PBS then incubated with secondary antibody HRP ferritin polyclonal (Abcam®; 1:2000 in diluent buffer). After one hour incubation at room temperature, plates were washed as above then 100 μl TMB solution added to the wells. Colorimetric development was performed at room temperature and stopped with 50 μl 1 M hydrochloric acid and absorbance read at 450 nm.

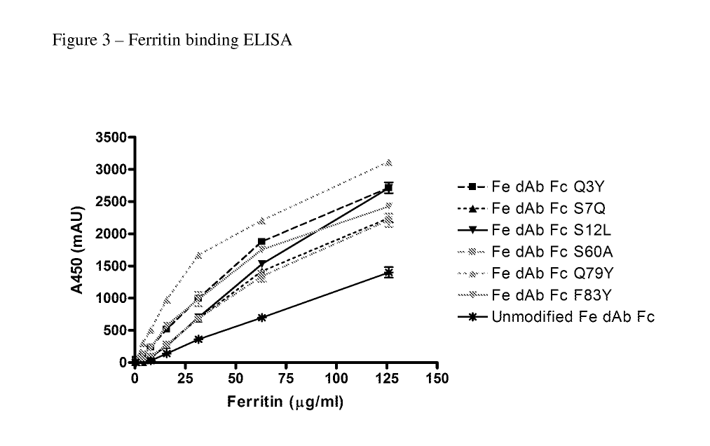

FIG. 3 illustrates the ability of the Ferritin dAb Fc proteins to bind human spleen ferritin, as assayed in the ferritin binding ELISA.

All Ferritin dAb Fc variants comprising the single framework substitutions were able to bind more ferritin than the unmodified Ferritin dAb Fc protein.

### BIBLIOGRAPHY

Ashkenazi et al. (1990). Mapping the CD4 binding site for human immunodeficiency virus by alanine-scanning mutagenesis. Proc Natl Acad Sci USA 87: 7150-7154
Barbas et al. (1992). Semisynthetic combinatorial antibody libraries: a chemical solution to the diversity problem. Proc Natl Acad Sci USA 89: 4457-4461
Barbas et al. (1992). Human monoclonal Fab fragments derived from a combinatorial library bind to respiratory syncytial virus F glycoprotein and neutralize infectivity. Proc Natl Acad Sci USA 89: 10164-10168.
Barderas et al. (2008). Affinity maturation of antibodies assisted by in silico modelling. Proc Natl Acad Sci USA 105: 9029-9034
Berzofsky et al. (1984). Antibody-Antigen Interactions. In Fundamental Immunology. Paul (Ed.) Raven Press: New York
Breitling et al. (1991). A surface expression vector for antibody screening. Gene 104: 147-153
Boder et al. (2000). Directed evolution of antibody fragments with monovalent femtomolar antigen-binding affinity. Proc Natl Acad Sci USA 97: 18870-18877
Burton et al. (1991). A large array of human monoclonal antibodies to type 1 human immunodeficiency virus from combinatorial libraries of asymptomatic seropositive individuals. Proc Natl Acad Sci USA 88:10134-10137
Cadwell and Joyce (1992). Randomization of genes by PCR mutagenesis. PCR Methods Appl. 2: 28-23
Caton and Koprowski (1990). Influenza virus hemagglutinin-specific antibodies isolated from a combinatorial expression library are closely related to the immune response of the donor. Proc Natl Acad Sci USA 87:6450-6454
Chang et al. (1991). Expression of antibody Fab domains on bacteriophage surfaces. Potential use for antibody selection. Journal of Immunology 147: 3610-3614
Chatellier et al. (1995). Codon-based combinatorial alanine scanning site-directed mutagenesis: design, implementation, and polymerase chain reaction screening. Analytical Biochemistry 229: 282-290
Chen et al. (1999). Selection and analysis of an optimized anti-VEGF antibody: crystal structure an affinity matured Fab in complex with antigen. Journal of Molecular Biology 293: 865-881
Clackson et al. (1991). Making antibody fragments using phage display libraries. Nature 352: 624-628
Chothia et al. (1989). Conformations of immunoglobulin hypervariable regions. Nature 342: 877-883
Chothia and Lesk (1987). Canonical structures for the hypervariable regions of immunoglobulins. Journal of Molecular Biology 196: 901-917
Chen et al. (1999). Selection and analysis of an optimized anti-VEGF antibody: crystal structure of an affinity-matured fab in complex with antigen. Journal of Molecular Biology 293: 865-881
Coia et al. (2001). Protein affinity maturation involving E. coli mutator cells. Journal of Immunological Methods 251: 181-193
Cunnighman and Wells (1989). High-resolution epitope mapping of hGH-receptor interactions by alanine-scanning mutagenesis. Science 244: 1081-1085
Daugherty et al. (1998). Antibody affinity maturation using bacterial surface display. Protein Engineering 11: 825-832
de Kruif et al. (1995). Rapid selection of cell subpopulation-specific human monoclonal antibodies from a synthetic phage antibody library. Proc Natl Acad Sci USA 92: 3938-3942
Dooley and Flajnik (2005). Shark immunity bites back: affinity maturation and memory response in the nurse shark, Ginglymostoma cirratum. European Journal of Immunology 35:936-945.
Ellington and Szostak (1990). In vitro selection of RNA molecules that binding specific ligands. Nature 346: 818-822
Griffiths et al. (1994). Isolation of high affinity human antibodies directly from large synthetic repertoires. EMBO J. 13: 3245-3260
Finlay et al. (2009). Affinity maturation of a humanized rat antibody for anti-RAGE therapy: comprehensive mutagenesis reveals a high level of mutational plasticity both inside and outside the complementarity-determining regions. Journal of Molecular Biology 388: 541-558
Fischmann et al. (1991). Crystallographic refinement of the three-dimensional structure of the FabD1.3-lysozyme complex at 2.5-A resolution. Journal of Biological Chemistry 266: 12915-12920
Foote and Winter (1992). Antibody framework residues affecting the conformation of the hypervariable loops. Journal of Molecular Biology 224: 487-499
Fromant et al. (1995). Direct random mutagenesis of gene sized DNA fragments using polymerase chain reaction. Analytical Biochemistry 224: 347-353
Hamers-Casterman et al. (1993). Naturally occurring antibodies devoid of light chains. Nature 363: 446-448
Harayama (1998). Artificial evolution by DNA shuffling. Trends in Biotechnology 16: 258-261
Hawkins et al. (1992). Selection of phage antibodies by binding affinity. Mimicking affinity maturation. Journal of Molecular Biology 226: 889-896
Hawkins and Winter (1992). Cell selection strategies for making antibodies from variable gene libraries: trapping the memory pool. European Journal of Immunology 22: 867-870
Higgins et al. (1994). CLUSTAL W: improving the sensitivity of progressive multiple sequence alignment through sequence weighting, position-specific gap penalties and weight matrix choice. Nucleic Acids Research 22: 4673-4680
Ho et al. (2005). In vitro antibody evolution targeting germline hot spots to increase activity of an anti-CD22 immunotoxin. Journal of Biological Chemistry 280: 607-617
Holliger et al. (1993). “Diabodies”: small bivalent and bispecific antibody fragments. Proc Natl Acad Sci USA 90: 6444-6448
Holt et al. (2003). Domain antibodies: proteins for therapy. Trends in Biotechnology 21: 484-490
Honegger and Pluckthun (2001). Yet another numbering scheme for immunoglobulin variable domains: an automatic modelling and analysis tool. Journal of Molecular Biology 309: 657-670
Hoogenboom and Winter (1992). By-passing immunisation. Human antibodies from synthetic repertoires of germline VH gene segments rearranged in vitro. Journal of Molecular Biology 227: 381-388
Hoogenboom et al. (1991). Multi-subunit proteins on the surface of filamentous phage: methodologies for displaying antibody (Fab) heavy and light chains. Nucleic Acids Research 19: 4133-4137
Huse et al. (1989). Generation of a large combinatorial library of the immunoglobulin repertoire in phage lambda. Science 246: 1275-1281
Irving et al. (1996). Affinity maturation of recombinant antibodies using E. coli mutator cells. Immunotechnology 2: 127-143
Jackson et al. (1995). In vitro antibody maturation. Journal of Immunology 154: 3310-3319
Johnson and Wu (2001) Kabat Database and its applications: future directions. Nucleic Acids Res. 29: 205-206.
Junginger et al. (1994). In Drug Permeation Enhancement. Hsieh (Eds.) Marcel Dekker Inc: New York 59-90
Kabat et al. (1983). Sequence of Proteins of Immunological Interest. US Department of Health and Human Services.
Kang et al. (1991a). Linkage of recognition and replication functions by assembling combinatorial antibody Fab libraries along phage surfaces. Proc Natl Acad Sci USA 88: 4363-4366
Kang et al. (1991b). Antibody redesign by chain shuffling from random combinatorial immunoglobulin libraries. Proc Natl Aca Sci USA. 88: 11120-11123
Kast and Hilvert (1997). 3D structural information as a guide to protein engineering using genetic selection. Current Opinion in Structural Biology 7: 470-479
Kettleborough et al. (1991). Humanization of a mouse monoclonal antibody by CDR-grafting: the importance of framework residues on loop conformation. Protein Engineering 4: 773-783
Knappick et al. (2000). Fully synthetic human combinatorial antibody libraries (HuCAL) based on modular consensus frameworks and CDRs randomized with trinucleotides. Journal of Molecular Biology 296: 57-86
Koide et al. (2007). Exploring the capacity of minimalist protein interfaces: interface energetics and affinity maturation to picomolar KD of a single-domain antibody with a flat paratope. Journal of Molecular Biology 373: 941-953
Kuby (1992). Immunology. Freeman and Company: New York
Jones (1848). On a new substance occurring in the urine of a patient with “mollities ossium.” Phil. Trans Roy Soc London 55-62
Jones et al. (1986). Replacing the complementarity-determining regions in a human antibody with those from a mouse. Nature 321: 522-525
Lee et al. (2004). High-affinity human antibodies from phage-displayed synthetic Fab libraries with a single framework scaffold. Journal of Molecular Biology 340: 1073-1093
LeFranc (2003). IMGT, the international ImMunoGeneTics Database®. Nucleic Acids Research. Nucleic acids Research 31: 307-310
Low et al. (1996). Mimicking somatic hypermutation: affinity maturation of antibodies displayed on bacteriophage using a bacterial mutator strain. Journal of Molecular Biology 260: 359-368
Lowman et al. (1991). Selecting high-affinity binding proteins by monovalent phage display. Biochemistry 30: 10832-10838.
Marks et al. (1991). By-passing immunization. Human antibodies from V-gene libraries displayed on phage. Journal of Molecular Biology 222: 581-597
Marks et al. (1992). Molecular evolution of proteins on filamentous phage. Mimicking the strategy of the immune system. Journal of Biological Chemistry 267: 16007-160010
Marks (2004). Antibody affinity maturation by chain shuffling. Methods in Molecular Biology 248: 327-343
McCafferty et al. (1991). Phage-enzymes: expression and affinity chromatography of functional alkaline phosphatase on the surface of bacteriophage. Protein Engineering 4: 955-961
McCafferty et al. (1990). Phage antibodies: filamentous phage displaying antibody variable domains. Nature 348: 552-554
Mullinax et al. (1990). Identification of human antibody fragment clones specific for tetanus toxoid in a bacteriophage lambda immunoexpression library. Proc Natl Acad Sci USA 87: 8095-8099
Murase et al. (2003). EF-Tu binding peptides identified, dissected, and affinity optimized by phage display. Chemistry and Biology 10: 161-168
Muyldermans et al. (1994). Sequence and structure of VH domain from naturally occurring camel heavy chain immunoglobulins lacking light chains. Protein Engineering 7:1129-1135
Nissim et al. (1994). Antibody fragments from a ‘single pot’ phage display library as immunochemical reagents. EMBO J. 13: 692-698
Pal et al. (2005). Alternative views of functional protein binding epitopes obtained by combinatorial shotgun scanning mutagenesis. Protein Science 14: 2405-2413
Persson et al. (1991). Generation of diverse high-affinity human monoclonal antibodies by repertoire cloning. Proc Natl Acad Sci USA 88: 2432-2436
Qin et al. (2007). A novel domain antibody rationally designed against TNFα using variable region of human heavy chain antibody as scaffolds to display antagonistic peptides. Molecular Immunology 44: 2355-2361
Queen et al. (1989). A Humanized Antibody That Binds To The Interleukin 2 Receptor. Proc Natl Acad Sci USA 86: 10029-10033
Retter et al. (2005). VBASE2, an integrative V gene database. Nucleic Acids Res. 33: D671-4
Riechmann et al. (1988). Reshaping Human Antibodies For Therapy. Nature 332: 323-327
Ridgway et al. (1999). Identification of a Human anti-CD55 single-chain Fv by subtractive panning of a phage library using tumour and non tumour cell lines. Cancer Research 59: 2718-2723
Robinson (Ed.) (1994). Sustained and controlled release drug delivery systems. Marcel Dekker Inc: NY
Sali and Blundell (1993). Comparative protein modelling by satisfaction of spatial restraints. Journal of Molecular Biology 234: 779-815
Schier et al. (1996). Isolation of picomolar affinity anti-c-erbB-2 single-chain Fv by molecular evolution of the complementarity determining regions in the center of the antibody binding site. Journal of Molecular Biology 263: 551-567
Scott and Smith (1990). Searching for peptide ligands with an epitope library. Science 249: 386-390
Stemmer (1994). Rapid evolution of a protein in vitro by DNA shuffling. Nature 370: 389-391
Streltsov et al. (2004). Structural evidence for evolution of shark Ig new antigen receptor variable domain antibodies from a cell-surface receptor. Proc Natl Acad Sci USA 101:12444-12449.
Tawfik and Griffths (1998). Man-made cell-like compartments for molecular evolution. Nature Biotechnology 16: 652-656
Thom et al. (2006). Probing a protein-protein interaction by in vitro evolution. Proc Natl Acad Sci USA 103: 7619-7624
Thompson et al. (1996). Affinity maturation of a high-affinity human monoclonal antibody against the third hypervariable loop of human immunodeficiency virus: use of phage display to improve affinity and broaden strain reactivity. Journal of Molecular Biology 256: 77-88
Tuerk and Gold (1990). Systematic evolution of ligands by exponential enrichment: RNA ligands to bacteriophage T4 DNA polymerase. Science 249: 505-510
Tulip et al. (1992). Refined crystal structure of the influenza virus N9 neuraminidase-NC41 Fab complex. Journal of Molecular Biology 227: 122-148
Vajdos et al. (2002). Comprehensive functional maps of the antigen-binding site of an anti-ErbB2 antibody obtained with shotgun scanning mutagenesis. Journal of Molecular Biology 320: 415-428
Valjakka et al. (2002). Crystal structure of an in vitro affinity- and specificity-matured anti-testosterone Fab in complex with testosterone. Improved affinity results from small structural changes within the variable domains. Journal of Biological Chemistry 277: 44021-44027
van den Beucken et al. (2003). Affinity maturation of Fab antibody fragments by fluorescent-activated cell sorting of yeast-displayed libraries. FEBS letters 546: 288-294
Vaughan et al. (1996). Human antibodies with sub-nanomolar affinities isolated from a large non-immunized phage display library. Nature Biotechnology 14: 309-314
Verhoeyen et al. (1988). Reshaping human antibodies: grafting an antilysozyme activity. Science 239: 1534-1536
Ward et al. (1989). Binding activities of a repertoire of single immunoglobulin variable domains secreted from Escherichia coli. Nature 341: 544-546
Weiss et al. (2000). Rapid mapping of functional protein epitopes by combinatorial alanine-scanning. Proc Natl Acad Sci USA 97: 8950-8954
Wu et al. (1998). Stepwise in vitro affinity maturation of Vitaxin, an alphav beta3-specific humanized mAb. Proc. Natl. Acad. Sci. U.S.A. 95: 6037-6042
Xiang et al. (1999). Light-chain framework region residue Try71 of chimeric B72.3 antibody plays an important role in influencing TAG72 antigen binding. Protein Engineering 12: 417-421
Yang et al. (1995). CDR walking mutagenesis for the affinity maturation of a potent human anti-HIV-1 antibody into the picomolar range. Journal of Molecular Biology 254: 392-403
Yau et al. (2005). Affinity maturation of a V(H)H by mutational hotspot randomization. Journal of Immunological Methods 297: 213-224
Yelton et al. (1995). Affinity maturation of the BR96 anti-carcinoma antibody by codon-based mutagenesis. Journal of Immunology 155: 1994-2004
Yoon et al. (2006). Construction, affinity maturation, and biological characterization of an anti-tumour-associated glycoprotein-72 humanized antibody. Journal of Biological Chemistry 281: 6985-6992
Zha et al. (2003). Family shuffling with single-stranded DNA. Methods in Molecular Biology 182: 243-257
Zahnd et al. (2004). Directed in vitro evolution and crystallographic analysis of a peptide-binding single chain antibody fragment (scFv) with low picomolar affinity. Journal of Biological Chemistry 279: 18870-18877
Zhang et al. (2004). Improved breadth and potency of a HIV-1 neutralizing human single-chain antibody by random mutagenesis and sequence antigen panning. Journal of Molecular Biology 335: 209-219

1-33. (canceled)

34. A modified domain antibody in which the framework sequences of the unmodified domain antibody are as set out in SEQ ID NO 1 and wherein the modified domain antibody includes at least one substitution selected from the group consisting of D1A, D1Y, D1K, I2L, I2P, Q3Y, Q3F, Q3D, Q3H, S7A, S7G, S7D, S7Q, S7N, S9A, S10A, S10L, L11Q, S12A, S12L, S12V, S12T, S12Q, S14A, D17Y, D17H, R18D, R185, T20Y, T22A, T22Y, T22Q, T22H, T22K, Q38H, P40D, K42A, K425, K42H, P44L, K45S, K45L, K45Y, K45D, K45H, 148A, Y49S, Y49D, Y49H, G57A, G57S, G57L, G57Y, G57Q, G57H, G57K, P59A, P59S, P59D, P59H, S60A, S60G, S60L, S60Y, S60D, S60Q, S60H, S60P, S60K, R61H, F62Y, T69D, D70Q, L79A, L79G, L79S, L79Y, L79V, L79D, L79H, L79K, F83A, F83S, F83L, F83Y, F83D and conservative substitutions thereof and combinations thereof.

35. A modified domain antibody as claimed in claim 34 which comprises a substitution selected from the group consisting of L79G, L79S, L79Y, L79K, L79D, L79A, L79V and L79H.

36. A modified domain antibody as claimed in claim 34 which comprises a substitution selected from the group consisting of S60G, S60A, S60D, S60L, S60H, S60Q, S60Y, S60P and S60K.

37. A modified domain antibody as claimed in claim 34 which comprises a substitution selected from the group consisting of S12T, S12L, S12V, S12A and S12Q.

38. A modified domain antibody as claimed in claim 34 which comprises a substitution selected from the group consisting of F83Y, F83L, F83A, F83S and F83D.

39. A modified domain antibody as claimed in claim 34 which comprises a substitution selected from the group consisting of P59A, P59S, P59D and P59H.

40. A modified domain antibody as claimed in claim 34 which comprises a substitution selected from the group consisting of G57S, G57Q, G57H, G57A, G57L, G57Y and G57K.

41. A modified domain antibody as claimed in claim 34 which comprises a substitution selected from the group consisting of Q3F, Q3Y, Q3H and Q3D.

42. A modified domain antibody as claimed in claim 34 which comprises a substitution selected from the group consisting of S7A, S7N, S7G, S7Q and S7D.

43. A domain antibody as claimed in claim 34 in which the domain antibody is attached to an Fc or modified Fc.

44. A domain antibody as claimed in claim 43 in which the sequence of the modified Fc is SEQ ID NO 6 or SEQ ID NO 7.

45. A nucleic acid molecule encoding the domain antibody of claim 34.

46. A nucleic acid molecule as claimed in claim 45 in which the nucleic acid molecule has a sequence selected from SEQ ID NO. 8 to SEQ ID NO. 708.

47. A transformed cell comprising the nucleic acid molecule of claim 45 wherein the cell expresses the nucleic acid molecule.

48. A domain antibody which binds TNFα, the domain antibody having SEQ ID NO 1 wherein at least one amino acid in the framework regions is substituted, wherein the domain antibody has improved TNFα neutralising potency in comparison to the domain antibody of SEQ ID NO 1.

49. A domain antibody as claimed in claim 48 in which the substitution is selected from the group consisting of D1A, D1Y, D1K, I2L, I2P, Q3F, Q3Y, Q3D, Q3H, S7A, S7D, S7Q, S7G, S7N, S9A, S10A, S10L, S12A, S12L, S12Q, S12V, S12T, S14A, R18D, T22A, K42A, K45S, Y49H, G57S, G57Q, G57H, P59A, P59S, P59D, S60G, S60A, S60L, S60D, S60Q, S60H, F62Y, L79G, L79S, L79Y, L79D, L79K, F83A, F83L, F83S, F83Y and conservative substitutions thereof and combinations thereof.

50. A domain antibody as claimed in claim 49 which comprises a substitution selected from the group consisting of L79G, L79S, L79Y, L79K and L79D.

51. A domain antibody as claimed in claim 49 which comprises a substitution selected from the group consisting of S60G, S60A, S60D, S60L, S60H and S60Q.

52. A domain antibody as claimed in claim 49 which comprises a substitution selected from the group consisting of S12T, S12L, S12V, S12A and S12Q.

53. A domain antibody as claimed in claim 49 which comprises a substitution selected from the group consisting of F83Y, F83L, F83A and F83S.

54. A domain antibody as claimed in claim 49 which comprises a substitution selected from the group consisting of P59A, P59S and P59D.

55. A domain antibody as claimed in claim 49 which comprises a substitution selected from the group consisting of G57S, G57Q and G57H.

56. A domain antibody as claimed in claim 49 which comprises a substitution selected from the group consisting of Q3F, Q3Y, Q3H and Q3D.

57. A domain antibody as claimed in claim 49 which comprises a substitution selected from the group consisting of S7A, S7N, S7G, S7Q and S7D.

58. A domain antibody as claimed in claim 48 in which the domain antibody includes multiple substitutions.

59. A domain antibody as claimed in claim 58 in which the multiple substitutions are selected from the group consisting of S60A_F83Y, Q3Y_S12L, S12L_S60A_L79Y_F83Y, S7Q_S60A, S7Q_S12L, Q3Y_S7Q, S12L_L79Y_F83Y, Q3Y_S60A, S12L_S60A_F83Y, S7Q_L79Y_F83Y, Q3Y_S12L_S60A_F83Y, Q3Y_S7Q_S12L_S60A, Q3Y_S60A_F83Y, Q3Y_S7Q_S12L, Q3Y_S7Q_S12L_L79Y, Q3Y_S12L_L79Y, Q3Y_L79Y, S7Q_S12L_S60A_F83Y, L79Y_F83Y, Q3Y_S12L_L79Y_F83Y, S60A_L79Y_F83Y, S7Q_S12L_L79Y_F83Y, Q3Y_S7Q_S12L_S60A_L79Y_F83Y, Q3Y_S7Q_S60A, Q3Y_S7Q_S60A_L79Y and S12L_S60A_L79Y.

60. A domain antibody as claimed in claim 48 in which the domain antibody is attached to an Fc or modified Fc.

61. A domain antibody as claimed in claim 60 in which the sequence of the modified Fc is SEQ ID NO 6 or SEQ ID NO 7.

62. A nucleic acid molecule encoding the domain antibody of claim 48.

63. A nucleic acid molecule as claimed in claim 62 in which the nucleic acid molecule has a sequence selected from SEQ ID NO. 8 to SEQ ID NO. 708.

64. A transformed cell comprising the nucleic acid molecule of claim 62 wherein the cell expresses the nucleic acid molecule.

65. A domain antibody which binds TNFα, the domain antibody having SEQ ID NO 1 wherein at least one amino acid in the framework regions is substituted, wherein the domain antibody has improved TNFα binding and/or improved protein expression levels in comparison to the domain antibody of SEQ ID NO 1.

66. A domain antibody as claimed in claim 65 in which the substitution is selected from the group consisting of S7A, L11Q, D17Y, D17H, R18S, T20Y, T22Y, T22Q, T22H, T22K, Q38H, P40D, K425, K42H, P44L, K45L, K45Y, K45D, K45H, 148A, Y49S, Y49D, Y49H, G57A, G57S, G57L, G57Y, G57Q, G57H, G57K, P59S, P59D, P59H, S60A, S60L, S60Y, S60D, S60Q, S60H, S60P, S60K, R61H, T69D, D70Q, L79A, L79S, L79Y, L79D, L79H, L79K, F83A, F83Y, F83D and conservative substitutions thereof and combinations thereof.

67. A domain antibody as claimed in claim 65 in which the substitution is selected from the group consisting of S7A, Y49H, G57S, G57Q, G57H, P59S, P59D, S60A, S60L, S60D, S60Q, S60H, L79S, L79Y, L79D, L79K, F83A, F83Y and conservative substitutions thereof and combinations thereof.

68. A domain antibody as claimed in claim 65 in which the domain antibody is attached to an Fc or modified Fc.

69. A domain antibody as claimed in claim 68 in which the sequence of the modified Fc is SEQ ID NO 6 or SEQ ID NO 7.

70. A nucleic acid molecule encoding the domain antibody of claim 65.

71. A nucleic acid molecule as claimed in claim 70 in which the nucleic acid molecule has a sequence selected from SEQ ID NO. 8 to SEQ ID NO. 708.

72. A transformed cell comprising the nucleic acid molecule of claim 70 wherein the cell expresses the nucleic acid molecule.

73. A VL domain antibody comprising a VL framework acceptor sequence wherein the framework sequence has been modified such that the framework comprises at least one amino acid substitution at least one position selected from the group consisting of 3, 7, 12, 60, 79, 83 and combinations thereof.

74. A VL domain antibody as claimed in claim 73 wherein the domain antibody comprises at least one framework substitution selected from the group consisting of Y at position 3, Q at position 7, L at position 12, A at position 60, Y at position 79, Y at position 83 and combinations thereof.

75. A domain antibody as claimed in claim 73 in which the domain antibody is attached to an Fc or modified Fc.

76. A domain antibody as claimed in claim 75 in which the sequence of the modified Fc is SEQ ID NO 6 or SEQ ID NO 7.
